(19) United States
(12) Patent Application Publication
(10) Pub. No.: US 2024/0122928 A1
(43) Pub. Date: Apr. 18, 2024

(54) Methods For Treating Agitation In Subjects With Mild Cognitive Impairment Or Major Neurocognition Disorder

(71) Applicant: JANSSEN PHARMACEUTICA NV, Beerse (BE)

(72) Inventors: Wayne Drevets, San Diego, CA (US); Pascal Bonaventure, San Diego, CA (US); Gahan Pandina, Titusville, NJ (US)

(21) Appl. No.: 18/529,147

(22) Filed: Dec. 5, 2023

Related U.S. Application Data
(63) Continuation of application No. PCT/EP2023/057710, filed on Mar. 24, 2023.
(60) Provisional application No. 63/323,167, filed on Mar. 24, 2022.

(51) Int. Cl. A61K 31/506; A61P 25/28

(52) U.S. Cl. CPC A61K 31/506; A61P 25/28

(57) ABSTRACT

The present invention relates to a method of treating agitation in a human patient with dementia, comprising administering to the human patient with dementia in need thereof a pharmaceutically effective amount of a compound of formula (I) or a pharmaceutically acceptable salt or hydrate thereof to treat agitation, wherein R¹-R⁴ are defined herein:

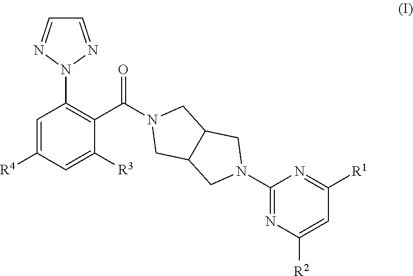

wherein the human patient has at least an about 20% reduction in their symptom of agitation, relative to the patient prior to administration of the compound of formula (I).

### CROSS-REFERENCE TO RELATED APPLICATIONS

This application is a continuation of International Patent Application No. PCT/EP2023/057710, filed Mar. 24, 2023, which claims the benefit of the priority of U.S. Provisional Patent Application No. 63/323,167, filed Mar. 24, 2022, the disclosures of which are incorporated by reference herein.

### TECHNICAL FIELD

The disclosure relates to methods of treating agitation in a human patient with dementia. This disclosure also relates to compounds for use in methods of treating agitation in a human patient with dementia, and to pharmaceutical compositions for use in methods of treating agitation in a human patient with dementia.

### BACKGROUND

Currently, approximately 50 million people worldwide are living with dementia and with nearly 10 million new cases diagnosed every year. Of all the dementias, Alzheimer's disease (AD) is the most common accounts for up to 70% of all dementia cases. AD is a progressive, neurodegenerative disorder affecting broad areas of the cerebral cortex and hippocampus. Even though psychopathology of AD can vary greatly from one person to another, it includes both common behavioral and psychological symptoms that are associated with profound distress for patients and caregivers, greater risk of institutionalization, and accelerated progression to severe dementia. AD is also a fatal disease. The three most common psychopathologies in AD are agitation, depression/affective symptoms, and psychosis.

As the population ages, the number of people with dementia will rise and a concomitant rise in the dependency ratio means that the economic burden of AD will increase dramatically. Especially in advanced stages of dementia, behavioral and psychological symptoms of dementia (BPSD) often lead to hospitalization or admission into long-term care facilities including nursing homes. The psychological symptoms that individuals with dementia (e.g. AD frontotemporal dementia, dementia with Lewy Bodies, poststroke dementia, and mixed Alzheimer's disease/vascular dementia) commonly experience are apathy, anhedonia, and poverty of behavioral retinue (amotivational syndrome) often associated with agitation. As dementia worsens cognitive abilities decline resulting in many individuals becoming confused, frustrated, irritable and unable to cope with change or situational factors resulting in many of these individuals being agitated. Agitation is one of the most commonly observed neuropsychiatric symptoms, found in up to 70% of dementia patients. Unfortunately, only a limited number of pharmacologic treatments for BPSD are approved in this population.

There are no efficacious treatments for the underlying causes of AD. Due to the variability in number, type and severity of BPSD between patients there are different symptomatic strategies for managing these patients, each with limitations and/or risks. Non-pharmacological approaches such as (psychological, psychosocial, behavioral or environmental interventions, complimentary therapies, and others) are used as first line treatments for behavioral symptoms associated with dementia.

Despite the absence of solid evidence about efficacy for the treatment of individual neuropsychiatric symptoms, several antipsychotics, antidepressants, mood stabilizers, anticonvulsants and benzodiazepines are used off-label to treat BPSD in people living with dementia (i.e. AD) when non-pharmacological approaches fail. These pharmacologic treatments are associated with high risk of occurrence of adverse effects and increased mortality, highlighting an ongoing and serious unmet medical need in this vulnerable patient population.

The present invention provides an effective method for treating agitation in patients with dementia. The method may comprise administering to the patient a pharmaceutically effective amount of a compound of formula (I):

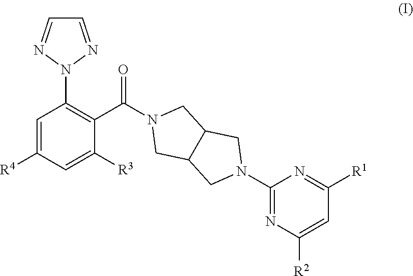

wherein:
R¹ is C₁₋₄ alkyl;
R² is C₁₋₄ alkyl;
R³ is H or halogen; and
R⁴ is H or C₁₋₄ alkoxy:
or a pharmaceutically acceptable salt or hydrate thereof.

### SUMMARY

The general description and the following detailed description are exemplary and explanatory only and are not restrictive of the disclosure, as defined in the appended claims. Other aspects of the present disclosure will be apparent to those skilled in the art in view of the detailed description of the disclosure as provided herein.

The present invention relates to a method of treating agitation in a human patient with dementia, comprising administering to the human patient with dementia in need thereof a pharmaceutically effective amount of a compound of formula (I):

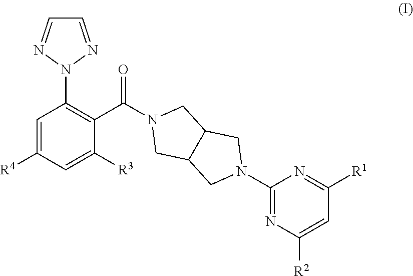

wherein:
R¹ is C₁₋₄ alkyl;
R² is C₁₋₄ alkyl;
R³ is H or halogen; and
R⁴ is H or C₁₋₄ alkoxy:
or a pharmaceutically acceptable salt or hydrate thereof to treat agitation wherein the human patients have at least an about 20% reduction in their symptom of agitation, relative to the patient prior to administration of the compound of formula (I).

In another aspect, this invention specifically relates to the treatment of patient with dementia selected from the group consisting of dementia from Alzheimer's disease, frontotemporal dementia, dementia with Lewy Bodies, poststroke dementia, and mixed Alzheimer's disease/vascular dementia. In a preferred aspect, this invention specifically relates to the treatment of patient with dementia from Alzheimer's disease.

In one preferred embodiment of the present invention, the human patients with Alzheimer's dementia have mild or moderate dementia.

In some embodiments, the compound is [5-(4,6-dimethyl-pyrimidin-2-yl)-hexahydro-pyrrolo[3,4-c]pyrrol-2-yl]-(2-fluoro-6-[1,2,3]triazol-2-yl-phenyl)-methanone or a pharmaceutically acceptable salt thereof. In yet other embodiments, the compound is (5-(4,6-dimethylpyrimidin-2-yl)hexahydropyrrolo[3,4-c]pyrrol-2(1H)-yl)(4-methoxy-2-(2H-1,2,3-triazol-2-yl)phenyl)methanone or a pharmaceutically acceptable salt thereof.

### BRIEF DESCRIPTION OF THE DRAWINGS

The present disclosure may be understood more readily by reference to the following detailed description taken in connection with the accompanying figures and examples, which form a part of this disclosure. The summary, as well as the following detailed description, is further understood when read in conjunction with the appended figures:

FIG. 1 is a bar graph showing body weights of mice prior to test. Data are presented as mean ±SEM. FIG. 1A is a bar graph showing body weights of mice prior to test. Data are presented as mean ±SEM.

FIG. 2 is a bar graph showing total distance traveled for each treatment group for Days 1, 5, 10, 11, and 15 AM session. Data are presented as mean ±SEM. One-way ANOVA followed by Fisher's LSD: *p<0.05, ***p<0.001 vs tTA-Vehicle; [symbol] p<0.0001 vs Tg4510-Vehicle. FIG. 2A is a bar graph showing total distance traveled for each treatment group for Days 1, 5, 10, 11, and 15 AM session. Data are presented as mean ±SEM. One-way ANOVA followed by Fisher's LSD: *p<0.05, ***p<0.001 vs tTA-Vehicle; [symbol] p<0.0001 vs Tg4510-Vehicle.

FIG. 3 are line graphs showing time course of distance traveled for each treatment group during the AM test sessions. Data are presented as mean ±SEM in 5 minute bins. FIG. 3A are line graphs showing time course of distance traveled for each treatment group during the AM test sessions. Data are presented as mean ±SEM in 5 minute bins.

FIG. 4 is a bar graph showing total distance traveled for each treatment group for Days 1, 5, 10, 11, and 15 PM session. Data are presented as mean ±SEM. One-way ANOVA followed by Fisher's LSD: **p<0.01 vs tTA-Vehicle; [symbol] p<0.05 vs Tg4510-Vehicle. FIG. 4A is a bar graph showing total distance traveled for each treatment group for Days 1, 5, 10, 11, and 15 PM session. Data are presented as mean ±SEM. One-way ANOVA followed by Fisher's LSD: **p<0.01 vs tTA-Vehicle; [symbol] p<0.05 vs Tg4510-Vehicle.

FIG. 5 are line graphs showing time course of distance traveled for each treatment group during the PM test sessions. Data are presented as mean ±SEM in 5 minute bins. FIG. 5A are line graphs showing time course of distance traveled for each treatment group during the PM test sessions. Data are presented as mean ±SEM in 5 minute bins.

FIG. 6 is a bar graph showing rearing episodes for each treatment group for Days 1, 5, 10, 11, and 15 AM session. Data are presented as mean ±SEM. One-way ANOVA followed by Fisher's LSD: *p<0.05 vs tTA-Vehicle; [symbol] p<0.05, [symbol] p<0.01, [symbol] p<0.001 vs Tg4510-Vehicle. FIG. 6A is a bar graph showing rearing episodes for each treatment group for Days 1, 5, 10, 11, and 15 AM session. Data are presented as mean ±SEM. One-way ANOVA followed by Fisher's LSD: *p<0.05 vs tTA-Vehicle; [symbol] p<0.05, [symbol] p<0.01, [symbol] p<0.001 vs Tg4510-Vehicle.

FIG. 7 are line graphs showing time course of rearing episodes for each treatment group during the AM test sessions. Data are presented as mean ±SEM in 5 minute bins. FIG. 7A are line graphs showing time course of rearing episodes for each treatment group during the AM test sessions. Data are presented as mean ±SEM in 5 minute bins.

FIG. 8 is a bar graph showing rearing episodes for each treatment group for days 1, 5, 10, 11, and 15 PM session. Data are presented as mean ±SEM. One-way ANOVA followed by Fisher's LSD: *p<0.05 vs tTA-Vehicle. FIG. 8A is a bar graph showing rearing episodes for each treatment group for days 1, 5, 10, 11, and 15 PM session. Data are presented as mean ±SEM. One-way ANOVA followed by Fisher's LSD: *p<0.05 vs tTA-Vehicle.

FIG. 9 are line graphs showing time course of rearing episodes for each treatment group during the PM test session. Data are presented as mean ±SEM in 5 minute bins. FIG. 9A are line graphs showing time course of rearing episodes for each treatment group during the PM test session. Data are presented as mean ±SEM in 5 minute bins.

FIG. 10 is the study scheme for Example 2. * actigraphy: worn continuously. ** All participants who discontinue study drug in the DB treatment phase will have an Early Withdrawal visit (Visit 6) and as a Follow-up visit (Visit 7). Participants who discontinue study drug prior to Day 35 may continue after the Follow-up visit (Visit 7) with additional follow-up visits every 2 weeks until Day 57. FIG. 10A is a study scheme for Example 2. * actigraphy: worn continuously. ** All participants who discontinue study drug in the DB treatment phase will have an Early Withdrawal visit (Visit 6) and as a Follow-up visit (Visit 7). Participants who discontinue study drug prior to Day 35 may continue after the Follow-up visit (Visit 7) with additional follow-up visits every 2 weeks until Day 57.

### DETAILED DESCRIPTION OF ILLUSTRATIVE EMBODIMENTS

### I. Definitions

Mild neurocognitive disorder or mild cognitive impairment (MCI) is a condition in which subjects demonstrate cognitive impairment with minimal or limited impairment of instrumental activities of daily living. Although it can be the first cognitive sign of Alzheimer's disease it can also be secondary to other disease processes (e.g. neurologic, other neurodegenerative disorders, systemic infectious or psychiatric disorders). DSM-5 provides two criteria for this diagnosis. Criterion A requires that there is evidence of a modest cognitive decline from a previous level of performance in 1 or more cognitive domain (complex attention, executive function, learning and memory, language, perceptual motor, or social cognition) based (1) on concern of the subject, a knowledgeable informant, or the clinician that there has been a significant decline in cognitive function; and (2) a substantial impairment in cognitive performance, preferably documented by standardized neuropsychological testing or in its absence, another quantified clinical assessment. Criterion B requires that the cognitive deficits do not interfere with capacity for independence in everyday activities (i.e. complex instrumental activities of daily living such as paying bills or managing medication are preserved, but greater effort, compensatory strategies, or accommodation may be required). Provided, that the cognitive deficits do not occur exclusively in the context of a delirium or the cognitive deficits are not better explained by another mental disorder (e.g. major depressive disorder or schizophrenia). Anywhere from between 3 to 13% of patients with mild neurocognitive disorder will progress to a major neurocognitive disorder (dementia) each year. However, not all individuals with MCI will go on to develop a dementia. MCI could be the prodromal state for an individual having Alzheimer's disease.

Major neurocognitive disorder (NCD) as defined in the DSM 5 is evidenced by a significant cognitive decline from a previous level of performance in one or more cognitive domains: learning and memory, language, executive function, complex attention, perceptual motor and social cognition. The evidence of decline is based on concern of the individual, a knowledgeable informant or a clinician that there has been a significant decline in cognitive function and a substantial impairment in cognitive performance, preferably documented by standardized neuropsychological testing or, in its absence, another quantified clinical assessment. The cognitive deficits from this decline must interfere with independence in everyday activities. At a minimum, assistance should be required with complex instrumental activities of daily living, such as paying bills or managing medications. These cognitive deficits must not occur exclusively in the context of a delirium or are not better explained by another mental disorder (e.g. major depressive disorder or schizophrenia). NCD is essentially a different label for dementia, and NCD and dementia will be used interchangeably in this specification.

MCI or NCD may be caused by a variety of disorders including Alzheimer's disease, frontotemporal neurocognitive disorder, neurocognitive disorder with Lewy Bodies (e.g. Diffuse Lewy body disease), Huntington disease, Multiple Sclerosis, Parkinson disease, Pick's disease, brain trauma (e.g. intracerebral hemorrhage, subarachnoid hemorrhage, subdural or epidural hematoma, concussion), cardiovascular disorders (e.g. multi-infarct dementia, stroke, heart infections and transient ischemic attack), infections and metabolic causes. In some embodiments, MCI or NCD is caused by Alzheimer's disease. In other embodiments, MCI or NC is caused by frontotemporal neurocognitive disorder, i.e., frontotemporal dementia. In further embodiments, MCI or NCD is caused by a neurocognitive disorder with Lewy Bodies (e.g. Diffuse Lewy body disease), i.e. dementia with Lewy Bodies. In yet other embodiments, MCI or NCD is caused by Huntington's disease. In still further embodiments, MCI or NCD is caused by multiple sclerosis. In other embodiments, MCI or NCD is caused by Parkinson disease. In further embodiments, MCI or NCD is caused by Pick's disease. In still other embodiments, MCI or NCD is caused by brain trauma (e.g. intracerebral hemorrhage, subarachnoid hemorrhage, subdural or epidural hematoma, concussion, stroke, i.e., poststroke dementia). In yet other embodiments, MCI or NCD is caused by cardiovascular disorders (e.g. multi-infarct dementia, stroke, heart infections and transient ischemic attack). In still further embodiments, MCI or NCD is caused by an infection. In other embodiments, MCI or NCD has a metabolic cause. In further embodiments, MCI or NCD is caused by Alzheimer's disease and vascular issues, i.e., mixed Alzheimer's disease/vascular dementia.

Alzheimer's disease (AD) is typically characterized by level of symptomatic severity and impairment. AD is characterized by early, middle and late stages, corresponding to mild, moderate and severe which describe the extent of cognitive and behavioral symptoms and functional decline. There are five recognized stages of AD. The first two — pre-clinical and Mild Cognitive Impairment due to AD — are mainly utilized for research. Preclinical AD describes a long pre-symptomatic period, during which no clinical symptoms are detected. MCI due to AD is characterized by subtle memory deficits: memory symptoms may not be easily recognized, and there is little or no functional impact of any symptoms. Many patients also have MCI that is not due to AD, so this diagnosis is also typically used in a research context. When cognitive symptoms become recognizable, the first clinical diagnosis of Mild AD may be made upon clinical exam and cognitive testing. Mild memory symptoms, difficulty with complex tasks and organizing thoughts, distress or agitation stemming in part from cognitive and mood symptoms are common during this stage. Patients with mild AD live independently and can function day to day without support. In Moderate stage AD, memory symptoms become more pronounced, judgment becomes poor, and assistance is needed with some daily living tasks, along with personality changes. Moderate AD is typically the longest clinical stage, with functional decline becoming more evident with passing years. The final stage—Severe AD—is characterized by profound memory problems and behavioral changes. Substantial assistance with self-care is required, and patients may be unable to communicate. Ultimately, AD is fatal. Agitation is noted to occur across the spectrum of AD severity. Agitation in those with mild AD has been noted as a precursor or predictor of future AD diagnosis, and poorer prognosis.

Agitation in dementia patients is commonly characterized by motor restlessness, heightened responsivity to stimuli, inappropriate and/or purposeless verbal or motor activity. The symptoms fluctuate over time. Restlessness is often manifested by pacing, rocking, gesturing, pointing fingers, and performing repetitious mannerisms.Inappropriate verbal activities can include repetitive sentences and incessant phone calls, questions and strange noises. This diagnosis requires that the behavior is persistent or frequently recurrent for a minimum of two weeks and represents a change from the patient's usual behavior. The behavior must be severe enough to produce excess disability, in a clinician's opinion beyond that due to existing cognitive impairment, and includes at least one of the following:
Significant impairment in interpersonal relationships
Significant impairment in other aspects of social functioning
Significant impairment in ability to perform or participate in daily living activities.
   Additionally, while co-morbid conditions may be present, agitation is not attributable solely to another psychiatric disorder, suboptimal care conditions, medical condition, or the physiological effects of a substance. Improvement in agitation can be scored on any appropriate scale which separates agitation from aggression, such as the agitation specific score on the NPI-C scale.

Agitation and aggression are often confused in the literature but are two distinct syndromes. Aggression is overt behavior of humans involving intent to harm another or inanimate objects. Agitation is excessive motor or verbal activity without any focus or intent. In dementia patients, aggression in many circumstances may not linked to agitation, but arises independently as a result of mood dysregulation, paranoia, confusions as to third party motivation (i.e. fear of harm), lack of impulse control or delusional thinking. In those circumstances where agitation could escalate to aggression, appropriate treatment for agitation will reduce or prevent the escalation to aggression.

It is possible to separate patients with dementia and agitation or aggression into three groups of patients: those with only pure agitation, those with only pure aggression and those showing both agitation and aggression. In subjects with Alzheimer's disease, it is common for agitation to occur in even subjects with mild Alzheimer's dementia and its prevalence does not increase with dementia severity. Aggression starts to occur in the moderate stages of dementia and increases together with increasing communication deficit. Some subjects with dementia exhibit both agitation and aggression but typically not at the same time. Agitation is also associated with poor outcomes, including greater use of physical or mechanical restraints and psychotropic medications and institutionalization. Agitation—but not aggression—may be associated with increased mortality in later stages of AD.

Some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

As used herein, unless otherwise noted, the terms “treating”, “treatment” and the like, shall include the management and care of a subject or patient (preferably mammal, more preferably human) for the purpose of combating a disease, condition, or disorder and include the administration of a compound described herein to prevent the onset of the symptoms or complications, alleviate the symptoms or complications, or eliminate the disease, condition, or disorder. Similarly, “treatment” is used to encompass (a) reduction in the frequency of one or more symptoms; (b) reduction in the severity of one or more symptoms; (c) the delay or avoidance of the development of additional symptoms; and/or (d) delay or avoidance of the development of the disorder or condition, or any combination thereof

As used herein, unless otherwise noted, the terms “subject” and “patient” may be used interchangeably and refer to an animal, preferably a mammal, most preferably a human, who has been the object of treatment, observation or experiment. In some embodiments, the subject or patient has experienced and/or exhibited at least one symptom of the disease or disorder to be treated and/or prevented. One skilled in the art will further recognize that the methods of treatment are directed to subjects or patients in need of such treatment, prevention or dosing regimen, more particularly to subjects or patients diagnosed with or exhibiting at least one symptom of agitation regardless of type or underlying cause.

One skilled in the art will recognize that wherein methods of prevention are described, a subject in need thereof (i.e. a subject in need of prevention) shall include any subject who has experienced or exhibited at least one symptom of the disorder, disease or condition to be prevented. Further, a subject in need thereof may additionally be a subject (preferably a mammal, more preferably a human) who has not exhibited any symptoms of the disorder, disease or condition to be prevented, but who has been deemed by a physician, clinician or other medical profession to be at risk of developing said disorder, disease or condition. For example, the subject may be deemed at risk of having new episodes of agitation (and therefore in need of secondary prevention or preventive treatment) as a consequence of the subject's medical history, including, but not limited to, family history, pre-disposition, co-existing (comorbid) disorders or conditions, genetic testing, and the like.

Further, some of the quantitative expressions herein are recited as a range from about value X to about value Y. It is understood that wherein a range is recited, the range is not limited to the recited upper and lower bounds, but rather includes the full range from about value X through about value Y, or any value or range of values therein.

As used herein, the terms “including”, “containing” and “comprising” are used herein in their open, non-limiting sense.

### II. Method of Treatment

The present disclosure is related to the treatment agitation in subject having either MCI or NCD comprising administering a therapeutically effective amount of a selective orexin-2 antagonist of formula (I).

In Example 1, a selective orexin-2 antagonist was tested in a preclinical model of agitation. This model is used as a model for agitation without aggression observed in AD and other dementias. The mouse model (rTg4510) utilized a transgenic mouse carrying the human tau P301L tau mutation found in familial form of frontotemporal dementia. These mice in addition to having cognitive impairment exhibit a hyperactive phenotype which correlated with the progression of tau pathology and dependent on the P301L tau transgene expression (Jul et al. Journal of Alzheimer's Disease 49 (2016) 783-795). These mice display a hyperactive phenotype characterized by a significantly increased locomotor activity in a novel and in a simulated home cage environment together with a disturbed day/night cycle. The behavior of this mice correlates to some of the behavior disturbances observed in dementia such as is associated with advanced Alzheimer's disease and frontotemporal disease. The limiting factor on this experimental approach is animal habitation to the cage environment so the initial period of observation is most critical for understanding the effects of orexin-2 of the mice.

Turning to FIG. 2 (or FIG. 2A), on day 1 the AM phase is the normal sleep phase for these mice. The hyperlocomotion of the untreated transgenic mice is very apparent from graph. The normal mice and mice treated with the selective Orexin2 antagonist have much lower locomotion scores as compared to the untreated transgenic mice. The treated transgenic mice have a very similar level of locomotion to the tTA control mice as shown in distance traveled. The reduction in travel appears to be approximately 50% which correlates to a substantial reduction in agitation. On day 5 during the PM phase the active phase the selective orexin 2 antagonist increases locomotion in transgenic mice showing a corrective effect during the active phase.

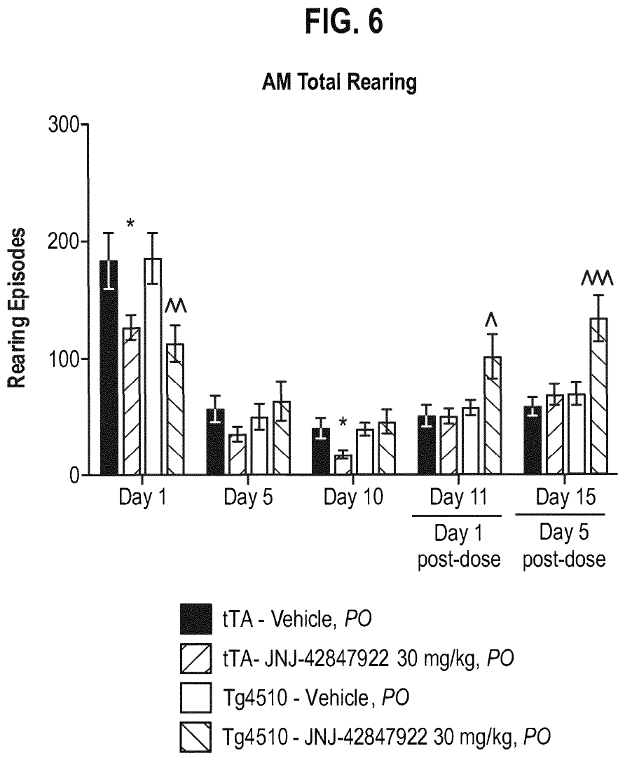

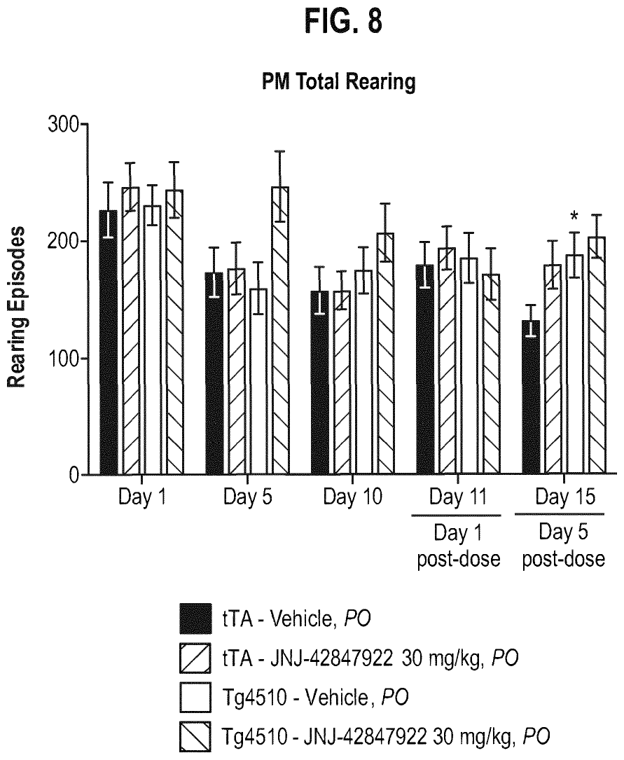

Although the current model of agitation being discussed is based on locomotion, it is noted that the rearing behavior seen in the treated mice bears commenting upon. In the later stage of treatment, the treated mice as shown in FIGS. 6 and 8 (or FIGS. 6A and 8A) had a tendency to rear up on their hind legs more often, while not being bound by this hypothesis, rearing is normally associated in with animals that are less anxious, which suggest the treated animals consistent with the locomotion findings, are rearing because they are less agitated.

Example 2 is a prophetic example of a human study which is expected to confirm the results seen in Example 1, as well as provide evidence of the additional benefits provided by the method of treatment described in this disclosure.

In one aspect, the present invention relates to the reduction of agitation in a human patient with dementia wherein the agitation is reduced by at least about 20%, preferably the agitation is reduced by at least about 25%, more preferably about 30%, most preferably by at least about 35% as determined on an appropriate agitation scale such as the NPI-C scale. The reduction of agitation is relative to the patient prior to administration of the compound of formula (I).

The NPI-C is an instrument developed on the basis of the original NPI that gives a score based on product of frequency and severity ratings of 12 symptom domains that can then be summed to a total score. For NPI-C, the items and domains of the original NPI have been expanded and the rating approach has been modified to include a clinician rating methodology. The rating methodology of the NPI-C incorporates the expert clinician's impressions to the data provided by the patients and caregivers.

The NPI-C can be used to rate the presence of NPS across many domains, as in the NPI-12, as a stand- alone measure for specific NPS domains (e.g., dysphoria, agitation), or a combination of both (presence of NPS across domains plus particular focus on one or more specific domains). For example, five domains may be measured with NPI-C, namely Delusions, Hallucinations, Agitation, Aggression, Dysphoria. This may include both caregiver and participant interviews. Regardless of whether the answer to the domain screening question is “Yes” or “No”, the NPI-C ratings for these domains may be done in their entirety (i.e. —questions asked and rated, with a corresponding Clinician Impression of Severity score).

In one aspect, the present invention relates to the reduction of agitation/aggression in a human patient with dementia, as determined on an appropriate scale, such as the sum of agitation and aggression domain scores (A+A) of the NPI-C. For example, the reduction of agitation, or the reduction of agitation/aggression, may be determined by a change from baseline to a second time point (e.g., at day 43) on the NPI-C A+A scores.

Other methods for determining the reduction of agitation, or the reduction of agitation/aggression, may include the Sleep Disorder Inventory (SDI) and the Cohen-Mansfield Agitation Inventory Community version (CMAI-C). For example, the SDI total score or the total CMAI-C score may be used to determine the reduction. For example, the reduction of agitation, or the reduction of agitation/aggression, may be determined by the CMAI-C total score, for example as determined by a change from baseline to a second time point (e.g., day 43) on the CMAI-C total score.

The CMAI-C is a 37-item scale that measures the ability of a drug to reduce overall frequency of agitation symptoms, including aggressive behaviors. Individual items are rated by an expert clinician on a scale of 1 to 7 in which a score of 7 represents the most frequent for each item assessed.

In another embodiment of the present invention, a reduction of one or more symptoms of agitation is seen after treatment with a selective orexin-2 antagonist wherein the symptoms is selected from the group consisting of motor restlessness, heightened responsivity to stimuli, inappropriate and/or purposeless verbal or motor activity, and/or fluctuation of symptoms over time. In some embodiments, the symptom is motor restlessness. In other embodiments, the symptom is heightened responsivity to stimuli. In further embodiments, the symptom is irritability. In yet other embodiments, the symptom is inappropriate and/or purposeless verbal or motor activity.

In one aspect, the present invention relates to a treatment for agitation that reduces or prevents the escalation to aggression. For example, the present invention may relate to a treatment for agitation that prevents the escalation to aggression.

In another embodiment of the present invention the treatment of MCI or NCD patients for agitation will reduce the incidence of agitated subjects progressing to aggressive subject as shown by an appropriate scale such as the NPI-C.

In another aspect, the present invention relates to the treatment of MCI or NCD in human subjects. Preferably the subject has MCI or NCD as a result of Alzheimer's disease, frontotemporal neurocognitive disorder, neurocognitive disorder with Lewy Bodies (e.g. Diffuse Lewy body disease), Huntington disease, Multiple Sclerosis, Parkinson disease Pick's disease, brain trauma (e.g. intracerebral hemorrhage, subarachnoid hemorrhage, subdural or epidural hematoma, concussion), cardiovascular disorders (e.g. multi-infarct dementia, stroke heart infections and transient ischemic attack), infections and metabolic causes. More preferably MCI or NCD is caused by a condition selected from the group consisting of Alzheimer's disease, frontotemporal dementia, diffuse Lewy Bodies, poststroke dementia, or mixed Alzheimer's disease/vascular dementia. Preferably, the MCI or NCD is caused by Alzheimer's disease. Preferably for the treatment of dementia in subjects with Alzheimer's disease the subjects have mild to moderate Alzheimer's disease.

In another aspect, the methods of treating a subject with dementia suffering from or diagnosed with agitation comprise administering to a subject in need of such treatment an effective amount of orexin-2 antagonist of formula (1).

The compound is preferably administered once daily and is administered to the subject prior to sleep. For example, the compound is administered within about 2 hours of sleep, within about 1 hour, or within about 30 minutes before sleep. In other embodiments, the compound is administered at least about 4 hours before the subject wakes or intends to wake from sleep, including about 5 hours, about 5.5 hours, about 6 hours, about 6.5 hours, about 7 hours, about 7.5 hours, about 8 hours, about 8.5 hours, about 9 hours, about 9.5 hours, about 10 hours, about 10.5 hours, about 11 hours, about 11.5 hours, or about 12 hours before the subject wakes or intends to wake from sleep. In certain embodiments, the compound is administered at least 6 hours to about 12 hours before the subject wakes or intends to wake from sleep. In preferred embodiments, the compound is administered at night.

After administration of the compound, the compound undergoes at least one half-life before the subject wakes from sleep. In other embodiments, the compound undergoes at least two half-lives, and preferably at least three half-lives before the subject wakes from sleep.

Therapeutically effective amounts for the compounds described herein include amounts that elicit the biological or medicinal response in a tissue system, animal or human that is being sought by a researcher, veterinarian, medical doctor, or other clinician, which includes alleviation of the symptoms of the disease or disorder being treated. Optimal dosages to be administered may be readily determined by those skilled in the art, and may vary with the mode of administration, the strength of the preparation and the advancement of the disease condition. Such factors including the particular patient being treated, including patient's sex, age, weight, diet, time of administration and concomitant diseases, among others. In certain embodiments, the effective amount of each dose of the compounds described herein is about 0.05 to about 100 mg of compound per kg of subject's body weight per day, or preferably about 0.05 to 1 mg/kg/day, in single or divided dosage units (examples of such dosage units include 2.5 mg, 5 mg, 10 mg, and 20 mg tablets). For a 70-kg human, an illustrative range for a suitable dosage amount is from about 0.05 to about 0.9 mg/day, or about 3.5 to about 63 mg/day.

The effective amount of the compound described herein may also be described without reference to the weight of the subject. Accordingly, the effective amount of the compound is about 10 to about 60 mg and preferably is about 10 to about 40mg. In some embodiments, the effective amount of the compound is about 5mg, 10 mg, about 15 mg, about 20 mg, about 25 mg, about 30 mg, about 35 mg, or about 40 mg, or within a range defined by any two of these values. In some embodiments, the effective amount is about 10 mg. In further embodiments, the effective amount is about 15 mg. In other embodiments, the effective amount is about 20 mg. In yet further embodiments, the effective amount is about 25 mg. In still other embodiments, the effective amount is about 30 mg. In further embodiments, the effective amount is about 35 mg. In other embodiments, the effective amount is about 40 mg. In yet further embodiments, the effective amount is about 10 to about 55, about 10 to about 50, about 10 to about 45, about 10 to about 40, about 10 to about 35, about 10 to about 30, about 10 to about 25, about 10 to about 20, about 10 to about 15, about 15 to about 60, about 15 to about 55, about 15 to about 50, about 15 to about 45, about 15 to about 40, about 15 to about 35, about 15 to about 30, about 15 to about 25, about 15 to about 20, about 20 to about 60, about 20 to about 55, about 20 to about 50, about 20 to about 45, about 20 to about 40, about 20 to about 35, about 20 to about 30, about 20 to about 25, about 25 to about 60, about 25 to about 55, about 25 to about 55, about 25 to about 50, about 25 to about 45, about 25 to about 40, about 25 to about 35, about 25 to about 30, about 30 to about 60, about 30 to about 55, about 30 to about 50, about 30 to about 45, about 30 to about 40, about 30 to about 35, about 35 to about 60, about 35 to about 55, about 35 to about 50, about 35 to about 45, about 35 to about 40, about 40 to about 60, about 40 to about 55, about 40 to about 50, about 40 to about 45, about 45 to about 60, about 45 to about 55, about 45 to about 50, about 50 to about 60, about 50 to about 55, or about 55 to about 60 mg.

Frequency adjustment can be accomplished by a one-time switch in frequency or may be determined over two or more administrations. By doing so, the attending physician or the like may determine an optimal frequency for administration and thereby tailor the administration to the patient.

One skilled in the art will recognize that in the methods described herein, the maintenance of the response in a patient may be determined by for example, a clinician, physician, psychiatrist, psychologist, or other suitable medical professional. Additionally, lack of symptoms of agitation, an absence of the need for additional or alternate treatment(s) for the agitation, or an absence of the worsening of the agitation. The physician or attending clinician may utilize any technique known in the art including, without limitation, general patient evaluation, diagnostic questionnaires, and evaluations such Neuropsychiatric Inventory Clinician (NPI-C).

### III. The Compounds

As discussed above, the compounds described herein are orexin-2 antagonists and may be used in the treatment of agitation in subjects having dementia. In some embodiments, the compounds are administered such that they have a time to maximal plasma concentration of less than about 3 hours, less than about 2 hours, and preferably less than about 1 hour, i.e., less than about 45 minutes, less than about 30 minutes, less than about 15 minutes, among others. In other embodiments, the compound has an elimination half-life of about 4 hours and typically less than about 4 hours. For example, certain compounds of the present disclosure have a half-life of about 2 to about 3 hours, e.g., about 2 hours, about 2.1 hours, about 2.2 hours, about 2.3 hours, about 2.4 hours, about 2.5 hours, about 2.6 hours, about 2.7 hours, about 2.8 hours, or about 2.9 hours to about 3 hours. Given the short half-life, the amount of the compound remaining in the subject upon waking is typically below the threshold required for pharmacodynamic effect.

In certain embodiments, the compound has the structure of formula (I):

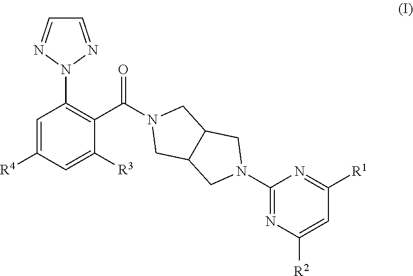

R¹ is C₁₋₄ alkyl. In some embodiments, R¹ is CH₃.

R² is C₁₋₄ alkyl. In some embodiments, R² is CH₃.

R³ is H or halogen. In some embodiments, R³ is halogen. In other embodiments, R³ is fluorine. In further embodiments, R³ is H.

R⁴ is H or C₁₋₄ alkoxy. In some embodiments, R⁴ is H. In further embodiments, R⁴ is C1-4alkoxy. In other embodiments, R⁴ is methoxy.

In some embodiments, R¹ is CH₃, R² is CH₃, R³ is fluorine or H, and R⁴ is H or methoxy.

“Alkyl” refers to a straight- or branched-chain alkyl group having from 1 to 12 carbon atoms in the chain. Examples of alkyl groups include methyl, ethyl, n-propyl, isopropyl, butyl, isobutyl, sec-butyl, tert-butyl and groups that in light of the ordinary skill in the art and the teachings provided herein would be considered equivalent to any one of the foregoing examples.

The term “cycloalkyl” refers to a saturated or partially saturated, monocyclic, fused polycyclic, or spiro polycyclic carbocycle having from 5 to 7 ring atoms per carbocycle. Illustrative examples of cycloalkyl groups include the following entities, in the form of properly bonded moieties:

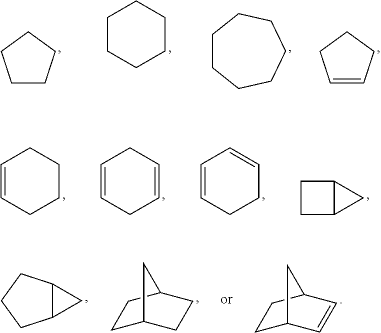

A “heterocycloalkyl” refers to a monocyclic ring structure that is saturated or partially saturated and has from 4 to 7 ring atoms per ring structure selected from carbon atoms and up to two heteroatoms selected from nitrogen, oxygen, and sulfur. The ring structure may optionally contain up to two oxo groups on sulfur ring members. Illustrative entities, in the form of properly bonded moieties, include:

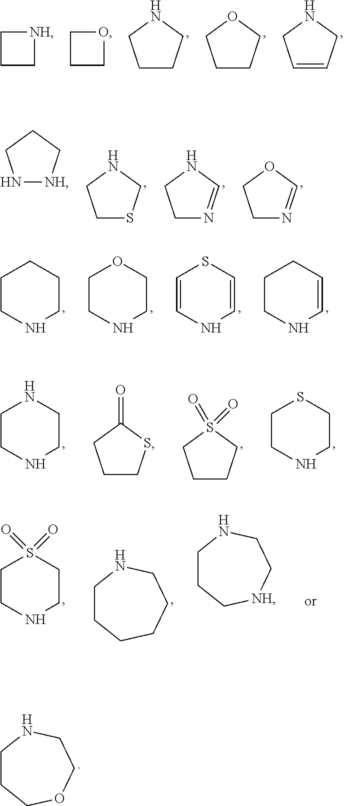

The term “heteroaryl” refers to a monocyclic, fused bicyclic, or fused polycyclic aromatic heterocycle (ring structure having ring atoms selected from carbon atoms and up to four heteroatoms selected from nitrogen, oxygen, and sulfur) having from 3 to 12 ring atoms per heterocycle. Illustrative examples of heteroaryl groups include the following entities, in the form of properly bonded moieties:

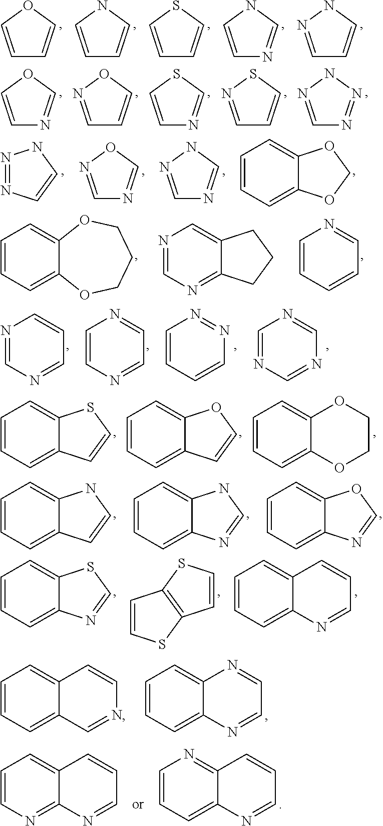

Those skilled in the art will recognize that the species of heteroaryl, cycloalkyl, and heterocycloalkyl groups listed or illustrated above are not exhaustive, and that additional species within the scope of these defined terms may also be selected.

“Alkoxy” includes a straight chain or branched alkyl group with a terminal oxygen linking the alkyl group to the rest of the molecule. Alkoxy includes methoxy, ethoxy, propoxy, isopropoxy, butoxy, t-butoxy, and so on.

“Halogen” represents chlorine, fluorine, bromine or iodine.

When referring to any formula given herein, the selection of a particular moiety from a list of possible species for a specified variable is not intended to define the same choice of the species for the variable appearing elsewhere. In other words, where a variable appears more than once, the choice of the species from a specified list is independent of the choice of the species for the same variable elsewhere in the formula, unless stated otherwise.

The nomenclature “Cᵢ₋ⱼ” with j>i, when applied herein to a class of substituents, is meant to refer to embodiments for which each and every one of the number of carbon members, from i to j including i and j, is independently realized. By way of example, the term C₁₋₃ refers independently to embodiments that have one carbon member (C₁), embodiments that have two carbon members (C₂), and embodiments that have three carbon members (C₃).

The term Cₙ₋ₘalkyl refers to an aliphatic chain, whether straight or branched, with a total number N of carbon members in the chain that satisfies n≤N≤m, with m>n.

Any formula given herein is intended to represent a compound having a structure depicted by the structural formula as well as certain variations or forms. In particular, a compound of any formula given herein may have asymmetric centers and therefore exist in different enantiomeric forms. All optical isomers and stereoisomers of the compounds of the general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers, or as atropisomers.

The compounds may include those described in U.S. Pat. No. 8,653,263 and US Patent Publication No. 2014/0171430, both of which are incorporated herein by reference. In some embodiments, the compound is [5-(4,6-dimethyl-pyrimidin-2-yl)-hexahydro-pyrrolo[3,4-c]pyrrol-2-yl]-(2-fluoro-6-[1,2,3]triazol-2-yl-phenyl)- methanone or a pharmaceutically acceptable salt or hydrate thereof. In some embodiments, the compound is [5-(4,6-dimethyl-pyrimidin-2-yl)-hexahydro-pyrrolo[3,4-c]pyrrol-2-yl]-(2-fluoro-6-[1,2,3]triazol-2-yl-phenyl)-methanone or a pharmaceutically acceptable salt thereof. In other embodiments, the compound is [5-(4,6-dimethyl-pyrimidin-2-yl)-hexahydro-pyrrolo[3,4-c]pyrrol-2-yl]-(2-fluoro-6-[1,2,3]triazol-2-yl-phenyl)-methanone. In further embodiments, the compound is [5-(4,6-dimethyl-pyrimidin-2-yl)-hexahydro-pyrrolo[3,4-c]pyrrol-2-yl]-(2-fluoro-6-[1,2,3]triazol-2-yl-phenyl)-methanone hydrochloride. In yet other embodiments, the compound is (5-(4,6-dimethylpyrimidin-2-yl)hexahydropyrrolo[3,4-c]pyrrol-2(1H)-yl)(4-methoxy-2-(2H-1,2,3-triazol-2-yl)phenyl)methanone or a pharmaceutically acceptable salt or hydrate thereof. In yet other embodiments, the compound is (5-(4,6-dimethylpyrimidin-2-yl)hexahydropyrrolo[3,4-c]pyrrol-2(1H)-yl)(4-methoxy-2-(2H-1,2,3-triazol-2-yl)phenyl)methanone or a pharmaceutically acceptable salt thereof. In still further embodiments, the compound is (5-(4,6-dimethylpyrimidin-2-yl)hexahydropyrrolo[3,4-c]pyrrol-2(1H)-yl)(4-methoxy-2-(2H-1,2,3-triazol-2-yl)phenyl)methanone. In certain embodiments, the compound is (5-(4,6-dimethylpyrimidin-2-yl)hexahydropyrrolo[3,4-c]pyrrol-2(1H)-yl)(4-methoxy-2-(2H-1,2,3-triazol-2-yl)phenyl)methanone hydrate. In other embodiments, the compound is (5-(4,6-dimethylpyrimidin-2-yl)hexahydropyrrolo[3,4-c]pyrrol-2(1H)-yl)(4-methoxy-2-(2H-1,2,3-triazol-2-yl)phenyl)methanone hydrochloride hydrate. In further embodiments, the compound is (5-(4,6-dimethylpyrimidin-2-yl)hexahydropyrrolo[3,4-c]pyrrol-2(1H)-yl)(4-methoxy-2-(2H-1,2,3-triazol-2- yl)phenyl)methanone hydrobromide hydrate.

Additionally, any formula given herein is intended to refer also to hydrates, solvates, and polymorphs of such compound, and mixtures thereof, even if such forms are not listed explicitly. A compound of Formula (I) or pharmaceutically acceptable salts of a compound of Formula (I) may be obtained as solvates. Solvates include those formed from the interaction or complexation of a compound with one or more solvents, either in solution or as a solid or crystalline form. In some embodiments, the solvent is water and then the solvates are hydrates. In addition, crystalline forms of a compound of Formula (I) or pharmaceutically acceptable salts of a compound of Formula (I) may be obtained as co-crystals. In certain embodiments, a compound of Formula (I) is obtained in a crystalline form. In other embodiments, a crystalline form of a compound of Formula (I) is cubic in nature. In other embodiments, pharmaceutically acceptable salts of compounds of Formula (I) are obtained in a crystalline form. In still other embodiments, compounds of Formula (I) are obtained in one of several polymorphic forms, as a mixture of crystalline forms, as a polymorphic form, or as an amorphous form. In other embodiments, a compound of Formula (I) converts in solution between one or more crystalline forms and/or polymorphic forms.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds described herein include isotopes of hydrogen, carbon, nitrogen, oxygen, phosphorus, fluorine, and chlorine, such as ²H, ³H, ¹¹C, ¹³C, ¹⁴C, ¹⁵N, ¹⁸O, ¹⁷O, ³²P, ³²P, ³⁵S, ¹⁸F, ³⁶CL, ¹²⁵I, respectively. Such isotopically labeled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. In particular, an ¹⁸F or ¹¹C labeled compound may be particularly preferred for PET or an I¹²³ for SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds described herein and prodrugs thereof can generally be prepared by carrying out the procedures disclosed in the schemes or in the examples and preparations described below by substituting a readily available isotopically labeled reagent for a non-isotopically labeled reagent.

Also included are pharmaceutically acceptable salt of a compound of Formula (I) and of the specific compounds exemplified herein, and methods of treatment using such salts.

A “pharmaceutically acceptable salt” is intended to mean a salt of a free acid or base of a compound represented by Formula (I) that is non-toxic, biologically tolerable, or otherwise biologically suitable for administration to the subject. See, generally, G. S. Paulekuhn, “Trends in Active Pharmaceutical Ingredient Salt Selection based on Analysis of the Orange Book Database”, J. Med. Chem., 2007, 50:6665-72, S. M. Berge, “Pharmaceutical Salts”, J Pharm Sci., 1977, 66:1-19, and Handbook of Pharmaceutical Salts, Properties, Selection, and Use, Stahl and Wermuth, Eds., Wiley-VCH and VHCA, Zurich, 2002. Examples of pharmaceutically acceptable salts are those that are pharmacologically effective and suitable for contact with the tissues of patients without undue toxicity, irritation, or allergic response.

Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, y-hydroxybutyrates, glycolates, tartrates, methane-sulfonates, propanesulfonates, naphthalene-l-sulfonates, naphthalene-2-sulfonates, and mandelates. For example, the pharmaceutically acceptable salts may include chlorides or bromides.

The desired pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid, glutaric acid, or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, ethanesulfonic acid, any compatible mixture of acids such as those given as examples herein, and any other acid and mixture thereof that are regarded as equivalents or acceptable substitutes in light of the ordinary level of skill in this technology. For example, the pharmaceutically acceptable salt may be prepared by treatment of the free base with hydrochloric acid or hydrobromic acid.

When the compound of Formula (I) is an acid, such as a carboxylic acid or sulfonic acid, the desired pharmaceutically acceptable salt may be prepared by any suitable method, for example, treatment of the free acid with an inorganic or organic base, such as an amine (primary, secondary, or tertiary), an alkali metal hydroxide, alkaline earth metal hydroxide, any compatible mixture of bases such as those given as examples herein, and any other base and mixture thereof that are regarded as equivalents or acceptable substitutes in light of the ordinary level of skill in this technology. Illustrative examples of suitable salts include organic salts derived from amino acids, such as N-methyl-D-glucamine, lysine, choline, glycine and arginine, ammonia, carbonates, bicarbonates, primary, secondary, and tertiary amines, and cyclic amines, such as tromethamine, benzylamines, pyrrolidines, piperidine, morpholine, and piperazine, and inorganic salts derived from sodium, calcium, potassium, magnesium, manganese, iron, copper, zinc, aluminum, and lithium.

Pharmaceutically acceptable prodrugs of a compound of Formula (I) and treatment methods employing such pharmaceutically acceptable prodrugs are also contemplated. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula (I). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

Exemplary prodrugs include compounds having an amino acid residue, or a polypeptide chain of two or more (e.g., two, three, or four) amino acid residues, covalently joined through an amide or ester bond to a free amino, hydroxy, or carboxylic acid group of a compound of Formula (I). Examples of amino acid residues include the twenty naturally occurring amino acids, commonly designated by three letter symbols, as well as 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone.

Additional types of prodrugs may be produced, for instance, by derivatizing free carboxyl groups of structures of Formula (I) as amides or alkyl esters. Examples of amides include those derived from ammonia, primary C₁₋₆alkyl amines and secondary di(C₁₋₆alkyl) amines. Secondary amines include 5- or 6-membered heterocycloalkyl or heteroaryl ring moieties. Examples of amides include those that are derived from ammonia, C₁₋₃alkyl primary amines, and di(₁₋₂alkyl)amines. Examples of esters include C₁₋₇alkyl, C₁₋₇cycloalkyl, phenyl, and phenyl(C₁₋₆alkyl) esters. Preferred esters include methyl esters. Prodrugs may also be prepared by derivatizing free hydroxy groups using groups including hemisuccinates, phosphate esters, dimethylaminoacetates, and phosphoryloxymethyloxycarbonyls, following procedures such as those outlined in Fleisher, Adv. Drug Delivery Rev. 1996, 19, 115-130. Carbamate derivatives of hydroxy and amino groups may also yield prodrugs. Carbonate derivatives, sulfonate esters, and sulfate esters of hydroxy groups may also provide prodrugs. Derivatization of hydroxy groups as (acyloxy)methyl and (acyloxy)ethyl ethers, wherein the acyl group may be an alkyl ester, optionally substituted with one or more ether, amine, or carboxylic acid functionalities, or where the acyl group is an amino acid ester as described above, is also useful to yield prodrugs. Prodrugs of this type may be prepared as described in Robinson, J. Med. Chem. 1996, 39(I),10-18. Free amines can also be derivatized as amides, sulfonamides, or phosphonamides. All of these prodrug moieties may incorporate groups including ether (—O—), amine (—N—), and carboxylic acid (COO—) functionalities.

It is to be understood that this disclosure is not limited to the specific devices, methods, applications, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

### IV. Compositions

The compounds described herein, including the compounds of formula (I), may be formulated as a pharmaceutical composition to administration to a subject. Accordingly, a pharmaceutical composition may comprise (a) an effective amount of at least one compound described herein and (b) a pharmaceutically acceptable excipient. A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

Delivery forms of the pharmaceutical compositions containing one or more dosage units of the compounds described herein may be prepared using suitable pharmaceutical excipients and compounding techniques known or that become available to those skilled in the art. The compositions may be administered in the inventive methods by a suitable route of delivery, e.g., oral, parenteral, rectal, topical, ocular routes, or by inhalation.

The preparation may be in the form of tablets, capsules, sachets, dragees, powders, granules, lozenges, powders for reconstitution, or liquid preparations. In some embodiments, the compositions are formulated for intravenous infusion, topical administration, or oral administration. In certain embodiments, the compositions are formulated for immediate release.

For oral administration, the compounds can be provided in the form of tablets or capsules, or as a solution, emulsion, or suspension. In certain embodiments, the compounds may be taken with food.

Oral tablets may include a compound mixed with pharmaceutically acceptable excipients such as inert fillers, diluents, disintegrating agents, binding agents, lubricating agents, sweetening agents, flavoring agents, coloring agents, glidants and preservative agents. Suitable inert fillers include sodium and calcium carbonate, sodium and calcium phosphate, lactose, lactose monohydrate, starch, sugar, glucose, methyl cellulose, magnesium stearate, mannitol, sorbitol, hypromellose, and the like. Exemplary liquid oral excipients include ethanol, glycerol, water, and the like. Starch, polyvinyl-pyrrolidone, sodium starch glycolate, microcrystalline cellulose, crospovidone (cross-linked polyvinyl N-pyrrolidone or PVP), and alginic acid are suitable disintegrating agents. Binding agents may include hypromellose (hydroxypropyl methylcellulose or HPMC), starch and gelatin. The lubricating agent, if present, may be magnesium stearate, stearic acid, or talc. The glidant, if present, may be silica (SiO₂) such as colloidal silica. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, the compound may be mixed with a solid, semi-solid, or liquid diluent. Soft gelatin capsules may be prepared by mixing the compound with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions, or syrups or may be lyophilized or presented as a dry product for reconstitution with water or other suitable vehicle before use. Such liquid compositions may optionally contain pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The compounds described herein may also be administered by non-oral routes. For example, the compounds may be formulated for rectal administration. For parenteral use, including intravenous, intramuscular, or intraperitoneal routes, the compound may be provided in sterile aqueous solutions or suspensions, buffered to an appropriate pH and isotonicity or in parenterally acceptable oil. Suitable aqueous vehicles include Ringer's solution and isotonic sodium chloride. Such forms will be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses may range from about 1 to 1000 μg/kg/minute of the compound, admixed with a pharmaceutical carrier over a period ranging from several minutes to several days.

For topical administration, the compounds may be mixed with a pharmaceutical carrier at a concentration of about 0.1% to about 10% of drug to vehicle. Another mode of administering the compound may utilize a patch formulation to affect transdermal delivery.

Compounds may alternatively be administered by inhalation, via the nasal or oral routes, e.g., in a spray formulation also containing a suitable carrier.

### V. Kits

Also described herein are kits for administering one or more compounds described herein to a patient for the treatment of agitation in subjects with MCI or NCD (dementia). The representative kits include one or more dosage units comprising an effective amount of one or more compounds described herein for administration to a patient and at a given frequency.

The dosage unit may be formulated for delivery by any means. In certain embodiments, the dosage unit is formulated for oral, intravenous, intranasal, intramuscular, sublingual, transdermal, otic, or rectal delivery. In certain embodiments, the dosage unit is formulated for oral delivery.

The dosage unit may be formulated to contain any amount of a compound described herein, depending on the route of administration. Accordingly, each dosage unit may comprise the required dosage for the patient or may comprise a portion of a compound described herein which is required for a single dosage.

Also optionally included in the kits is a agitation symptom rating scale questionnaire. The questionnaire may be for use by the patient alone or in combination with a physician. The questionnaire may be useful for determining the level of agitation of the patient at any stage of compound administration. In one embodiment, the questionnaire is one or more of the questionnaires noted herein.

Instructions for performing the claimed methods and administering the compound may also be included in the kits described herein.

The kits may be organized to indicate a single formulation containing a compound described herein or combination of formulations, each containing a compound described herein. The composition may be sub-divided to contain appropriate quantities of a compound described herein. The unit dosage can be packaged compositions such as packeted powders, vials, ampoules, prefilled syringes, tablets, caplets, capsules, or sachets containing liquids.

The compound described herein may be a single dose or for continuous or periodic discontinuous administration. For continuous administration, a kit may include a compound described herein in each dosage unit. When varying concentrations of a compound described herein, the components of the composition containing the compound described herein, or relative ratios of the compound described herein or other agents within a composition over time is desired, a kit may contain a sequence of dosage units.

The kit may contain packaging or a container with a compound described herein formulated for the desired delivery route. The kit may also contain dosing instructions, an insert regarding the compound described herein, instructions for monitoring circulating levels of the compound, or combinations thereof. Materials for using the compound may further be included and include, without limitation, reagents, well plates, containers, markers, or labels, and the like. Such kits may be packaged in a manner suitable for treatment of a desired indication

Other suitable components to include in such kits will be readily apparent to one of skill in the art, taking into consideration the desired indication and the delivery route. The kits also may include, or be packaged with, instruments for assisting with the injection/administration of the compound to the patient. Such instruments include, without limitation, an inhalant, syringe, pipette, forceps, measuring spoon, eye dropper, or any such medically approved delivery means. Other instrumentation may include a device that permits reading or monitoring reactions in vitro.

The compound may be provided in dried, lyophilized, or liquid forms. When reagents or components are provided as a dried form, reconstitution generally is by the addition of a solvent. The solvent may be provided in another packaging means and may be selected by one skilled in the art.

A number of packages or kits are known to those skilled in the art for dispensing pharmaceutical agents. In certain embodiments, the package is a labeled blister package, dial dispenser package, or bottle.

Methods for optimizing a dosage of the compound for a patient having or being predisposed to agitation also are provided. These methods can include (a) administering an effective amount of the compound to the patient, (b) analyzing the effects of the compound, and (c) administering an effective amount of the compound to the patient less frequently of a defined duration.

The term “study intervention” throughout the protocol, refers to study drug (seltorexant or placebo).

Abbreviations and Definitions

| | |
|---|---|
| Aβ | Amyloid beta |
| AChE | Acetylcholinesterase |
| AE | Adverse event |
| AESI | Adverse event of special interest |
| AGP | Alpha-1-acid glycoprotein |
| ALT | Alanine aminotransferase |
| APOE | Apolipoprotein E |
| AST | Aspartate transaminase |
| BMI | Body mass index |
| BUN | Blood urea nitrogen |
| BPSD | Behavioral and Psychological Symptoms of |
| | Dementia |
| CrCl | Creatinine clearance |
| CDR | Clinical Dementia Rating |
| CMAI-C | Cohen-Mansfield Agitation Inventory community |
| | version |
| CPK | Creatine phosphokinase |
| CR | Copy reference |
| C-SSRS | Columbia Suicide Severity Rating Scale |
| DAT | Dementia of Alzheimer's type |
| DB | Double-blind |
| DSM-V | Diagnostic and Statistical Manual of Mental |
| | Disorders (5th edition) |
| DLBD | Diffuse lewy body dementia |
| ECG | Electrocardiogram |
| FSH | Follicle stimulating hormone |
| FT4 | Free thyroxine |
| FTD | Frontotemporal dementia |
| GGT | Gamma-glutamyltransferase |
| HIS | Hachinski Ischemic Scale |
| HbA1c | Hemoglobin A1c |
| HPA | Hypothalamic pituitary adrenal axis |
| ICF | Informed consent form |
| ICH | International Council on Harmonisation |
| IPA | International Psychogeriatric Association |
| IS | Insomnia symptoms |
| IWRS | Interactive web response system |
| LC-MS/MS | Liquid chromatography/mass spectrometry/mass |
| | spectrometry |
| LDH | Lactic acid dehydrogenase |
| MAOI | Monoamine oxidase inhibitor |
| MDD | Major depressive disorder |
| MDDIS | Major depressive disorder with insomnia symptoms |
| MI | Multiple imputation |
| MMRM | Mixed model repeated measures |
| MMSE | Mini-Mental State Examination |
| NfL | Neurofilament light chain |
| NPI | Neuropsychiatric Inventory |
| NPI-C | Neuropsychiatric Inventory-Clinician rating |
| NPS | Neuropsychiatric symptoms |
| OF | Open field |
| PD | Pharmacodynamic |
| PK | Pharmacokinetic |
| QTcF | QT interval corrected according to Fridericia's |
| | formula |
| SAE | Serious adverse event |
| SDI | Sleep disorder inventory |
| SOL | Sleep onset latency |
| TEAE | Treatment emergent adverse events |
| TSH | Thyroid-stimulating hormone |
| TST | Total sleep time |
| ULN | Upper limit of normal |
| VS | Vital signs |
| WBC | White blood cell |
| WASO | Wake after sleep onset |

### EXAMPLE 1

The goal of this study was to determine the efficacy of seltorexant on locomotor activity in Tg4510 mice. Seltorexant/JNJ-42847922 is [5-(4,6-dimethyl-pyrimidin-2-yl)-hexahydro-pyrrolo[3,4-c]pyrrol-2-yl]-(2-fluoro-6-[1,2,3]triazol-2-yl-phenyl)-methanone and has been since well before the priority date.

Female and male tTA and Tg4510 mice were purchased from Jackson Laboratory. Mice were group housed in Opti-MICE cages (Animal Care Systems, CO). All animals were examined and weighed prior to initiation of the study to assure adequate health and suitability. During the study, 12/12 light/dark cycles were maintained. The room temperature was maintained between 20 and 23° C. with a relative humidity maintained around 50%. Chow and water were provided ad libitum for the duration of the study. Baseline BW was recorded to balance animals into treatment groups. Animals in the same cage received the same treatment. Dosing commenced when mice were approximately 4 months of age. Tests were performed during the animal's light cycle and dark cycle phases as detailed below.

Seltorexant (30 mg/kg) was formulated in 30% sulfobutylether cyclodextrin and administered per os (PO) at a dose volume of 10 ml/kg. Seltorexant was administered once daily at the beginning of the light phase for 10 days.

Eight male and 8 female mice were assigned to each treatment group as follows:
1. tTA—Vehicle, PO (n=16)
2. tTA—seltorexant 30mg/kg, PO (n=16)
3. Tg4510—Vehicle, PO (n=15)*
4. Tg4510—seltorexant 30mg/kg, PO (n=16)
*One female Tg4510 animal was found dead due to unknown circumstances before dosing commenced.

### Locomotor Activity

Open Field was performed on Days 1, 5, 10, 11 and 15 immediately after dosing during the light cycle (morning session; AM) and again during the dark cycle (under red lights; afternoon session; PM). Animals were dosed daily for the first 10 days of the study. The open field tests on Day 11 and Day 15 occurred 1 and 5 days after the completion of dosing, respectively.

The OF chambers are Plexiglas square chambers (27.3×27.3×20.3 cm; Med Associates Inc., St Albans, VT) surrounded by infrared photo beams to measure horizontal and vertical activity. Mice were placed in the center of an open field activity box for 30 minutes. The OF chambers were cleaned in between each group of mice tested. Distance traveled was measured from horizontal beam breaks as the mouse moved.

Photocell beam interruptions were automatically recorded by a computer system. Data were captured from consecutive beam breaks in 5 minute bins. The time course for locomotor activity is presented in 5 minute bins during the 30 minute test. Total distance traveled is also summed during the 30 minutes test. Total and time course of rearing episodes are also presented as an additional measure.

### Statistical Analysis

Data from each OF session were analyzed by analysis of variance (ANOVA) followed by Fisher's least significant difference (LSD) post-hoc comparisons where appropriate. An effect was considered significant if p<0.05. Data are represented as the mean and standard error to the mean (s.e.m). Data points more the two standard deviations from mean were considered statistical outliers and were removed from the analysis.

### Results

Body weight

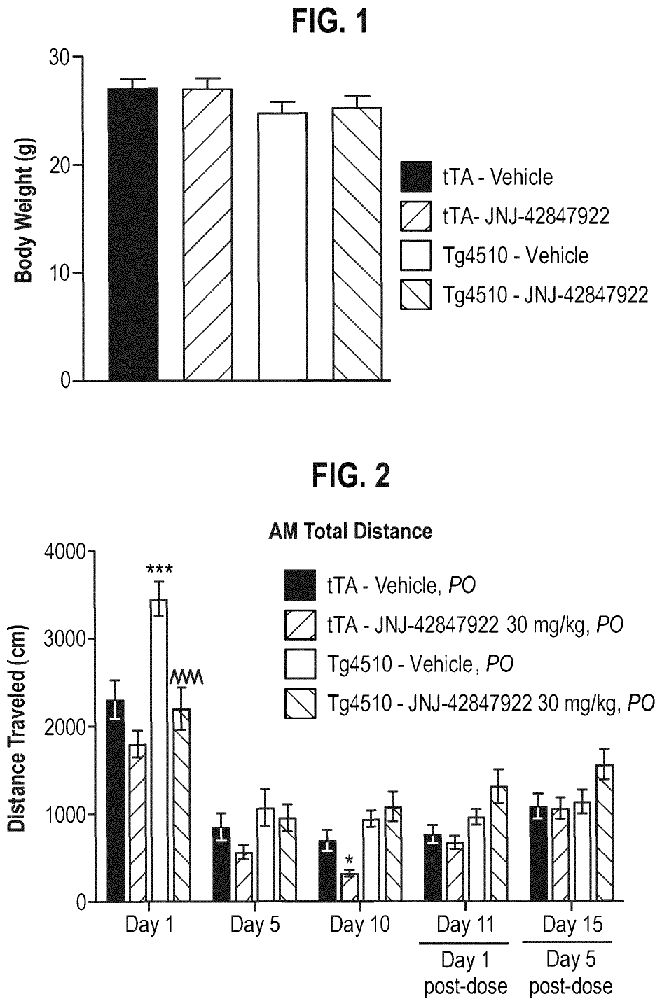

Baseline body weights of all mice, male and female combined, prior to test are shown in FIG. 1 (or FIG. 1A) and Table 1. One-way ANOVA found no significant difference between the treatment groups, indicating effective balancing into the groups.

TABLE 1
Body Weight

| ANOVA table | SS | DF | MS | F (DFn, DFd) | P value |
|---|---|---|---|---|---|
| Treatment | 71.84 | 3 | 23.95 | F (3, 59) = | P = 0.1931 |
| (between columns) | | | | 1.626 | |
| Residual | 869.0 | 59 | 14.73 | | |
| (within columns) | | | | | |
| Total | 940.8 | 62 | | | |

### Total Distance Traveled

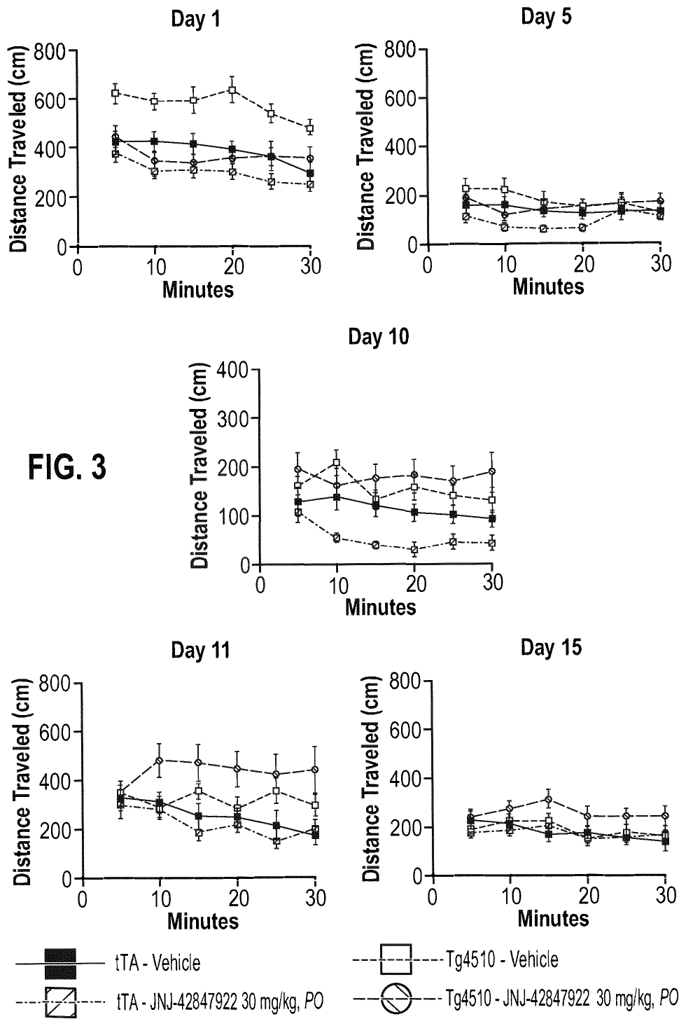

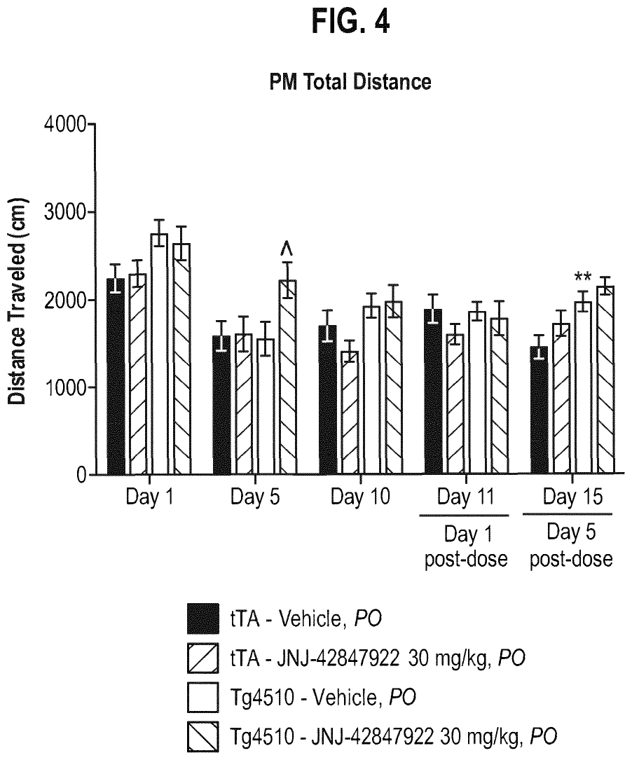

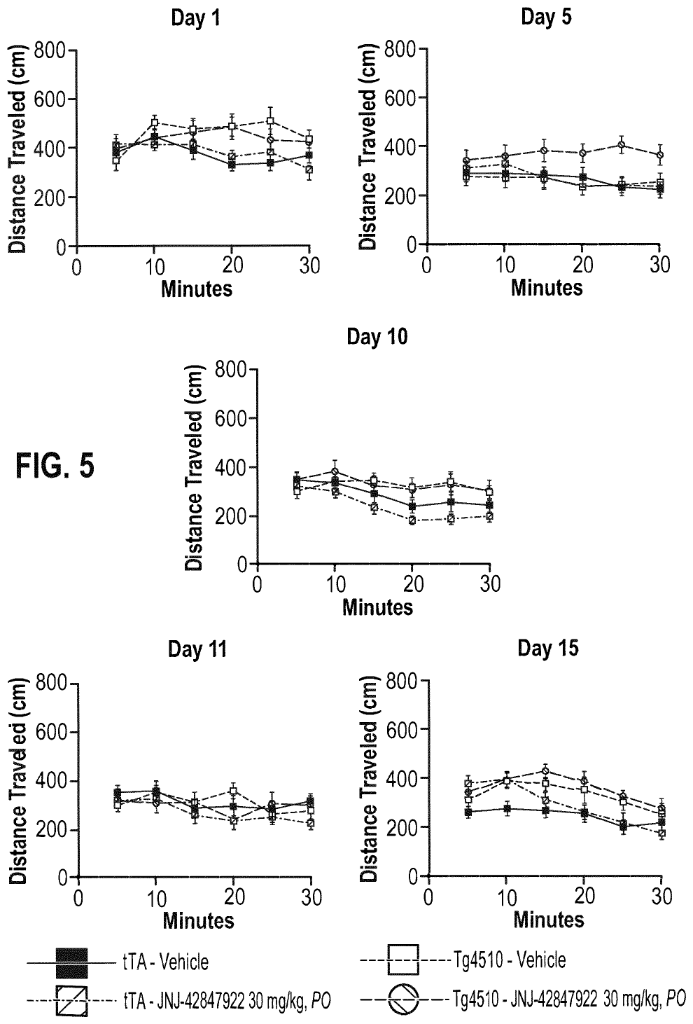

The total distance traveled by each group for Days 1, 5, 10, 11, and 15 is shown in FIG. 2 (or FIG. 2A) for the morning test sessions (AM) and in FIG. 4 (or FIG. 4A) for the afternoon test sessions (PM). Time course data are summarized in FIG. 3 (or FIG. 3A) (AM) and FIG. 5 (or FIG. 5A) (PM). One-way ANOVA conducted for each AM test session found a significant effect of Treatment on Days 1, 10, and 11 for the AM session. See, Table 2.

TABLE 2
Locomotor Activity - AM Test Sessions

| | | | | | |
|---|---|---|---|---|---|
| ANOVA table | SS | DF | MS | F (DFn, DFd) | P value |

Day 1

| | | | | | |
|---|---|---|---|---|---|
| Treatment (between columns) | 21712697 | 3 | 7237566 | F (3, 55) = | P < 0.0001 |
| Residual (within columns) | 34333221 | 55 | 624240 | 11.59 | |
| Total | 56045918 | 58 | | | |

Day 5

| | | | | | |
|---|---|---|---|---|---|
| Treatment (between columns) | 2139292 | 3 | 713097 | F (3, 56) = | P = 0.1177 |
| Residual (within columns) | 19511700 | 56 | 348423 | 2.047 | |
| Total | 21650992 | 59 | | | |

Day 10

| | | | | | |
|---|---|---|---|---|---|
| Treatment (between columns | 5042116 | 3 | 1680705 | F (3, 56) = | P = 0.0002 |
| Residual (within columns) | 11811612 | 56 | 210922 | 7.968 | |
| Total | 16853728 | 59 | | | |

Day 11

| | | | | | |
|---|---|---|---|---|---|
| Treatment (between columns) | 3728857 | 3 | 1242952 | F (3, 56) = | P = 0.0037 |
| Residual (within columns) | 13818628 | 56 | 246761 | 5.037 | |
| Total | 17547485 | 59 | | | |

Day 15

| | | | | | |
|---|---|---|---|---|---|
| Treatment (between columns) | 2446616 | 3 | 815539 | F (3, 55) = | P = 0.0588 |
| Residual (within columns) | 17020402 | 55 | 309462 | 2.635 | |
| Total | 19467018 | 58 | | | |

Fisher's LSD test on Day 1 AM found that Tg4510-Vehicle mice traveled significantly more than tTA-Vehicle mice (p<0.001) and that Tg4510- seltorexant 30 mg/kg treated mice traveled significantly less than Tg4510-Vehicle mice (p<0.0001). See, Table 3.

TABLE 3
Day 1 - AM

| | Mean | | Individual |
|---|---|---|---|
| Uncorrected Fisher's LSD | Diff. | Summary | P Value |
| tTA - Vehicle vs. Tg4510 - Vehicle | −1148 | *** | 0.0003 |
| tTA - Vehicle vs. tTA - | 508.7 | ns | 0.0834 |
| seltorexant 30 mg/kg | | | |
| Tg4510 - Vehicle vs. Tg4510 - | 1254 | **** | <0.0001 |
| seltorexant 30 mg/kg | | | |

Fisher's LSD test on Day 10 AM found that tTA-seltorexant 30 mg/kg treated mice traveled significantly less than tTA-Vehicle mice (p<0.05). See, Table 4.

TABLE 4
Day 10 - AM

| | Mean | | Individual |
|---|---|---|---|
| Uncorrected Fisher's LSD | Diff. | Summary | P Value |
| tTA - Vehicle vs. Tg4510 - Vehicle | −243.9 | ns | 0.1585 |
| tTA - Vehicle vs. tTA - | 374.2 | * | 0.0297 |
| seltorexant 30 mg/kg | | | |
| Tg4510 - Vehicle vs. Tg4510 - | −141.0 | ns | 0.4051 |
| seltorexant 30 mg/kg | | | |

Post-hoc comparisons conducted on Day 11 AM indicated no statistical difference between the groups. See, Table 5.

TABLE 5
Day 11 - AM

| | Mean | | Individual |
|---|---|---|---|
| Uncorrected Fisher's LSD | Diff. | Summary | P Value |
| tTA - Vehicle vs. Tg4510 - Vehicle | −198.8 | ns | 0.2861 |
| tTA - Vehicle vs. tTA - | 94.44 | ns | 0.6047 |
| seltorexant 30 mg/kg | | | |
| Tg4510 - Vehicle vs. Tg4510 - | −345.0 | ns | 0.0629 |
| seltorexant 30 mg/kg | | | |

One-way ANOVA on distance traveled for each PM test session found a significant effect of Treatment on Days 5 and 15. See, Table 6.

TABLE 6
Locomotor Activity - PM Test Sessions

| | | | | | |
|---|---|---|---|---|---|
| ANOVA table | SS | DF | MS | F (DFn, DFd) | P value |

Day 1

| | | | | | |
|---|---|---|---|---|---|
| Treatment (between columns) | 2940158 | 3 | 980053 | F (3, 58) = | P = 0.0787 |
| Residual (within columns) | 23869128 | 58 | 411537 | 2.381 | |
| Total | 26809286 | 61 | | | |

Day 5

| | | | | | |
|---|---|---|---|---|---|
| Treatment (between columns) | 4836911 | 3 | 1612304 | F (3, 58) = | P = 0.0447 |
| Residual (within columns) | 32717364 | 58 | 564092 | 2.858 | |
| Total | 37554275 | 61 | | | |

Day 10

| | | | | | |
|---|---|---|---|---|---|
| Treatment (between columns) | 3089597 | 3 | 1029866 | F (3, 58) = | P = 0.0587 |
| Residual (within columns) | 22730133 | 58 | 391899 | 2.628 | |
| Total | 25819730 | 61 | | | |

Day 11

| | | | | | |
|---|---|---|---|---|---|
| Treatment (between columns) | 782640 | 3 | 260880 | F (3, 56) = | P = 0.5251 |
| Residual (within columns) | 19396654 | 56 | 346369 | 0.7532 | |
| Total | 20179294 | 59 | | | |

Day 15

| | | | | | |
|---|---|---|---|---|---|
| Treatment (between columns) | 3986736 | 3 | 1328912 | F (3, 55) = | P = 0.0018 |
| Residual (within columns) | 12787851 | 55 | 232506 | 5.716 | |
| Total | 16774587 | 58 | | | |

Fisher's LSD test on Day 5 PM found that Tg4510-seltorexant 30 mg/kg treated mice traveled significantly more than Tg4510-Vehicle mice (p<0.05). See, Table 7.

TABLE 7
Day 5 - PM

| | Mean | | Individual |
|---|---|---|---|
| Uncorrected Fisher's LSD | Diff. | Summary | P Value |
| tTA - Vehicle vs. Tg4510 - Vehicle | 35.50 | ns | 0.8977 |
| tTA - Vehicle vs. tTA - | −18.89 | ns | 0.9435 |
| seltorexant 30 mg/kg | | | |
| Tg4510 - Vehicle vs. Tg4510 - | −668.1 | * | 0.0182 |
| seltorexant 30 mg/kg | | | |

Fisher's LSD test on Day 15 PM found that Tg4510-Vehicle mice traveled significantly more than tTA-Vehicle mice (p<0.01). See, Table 8.

TABLE 8
Day 15 - PM

| | Mean | | Individual |
|---|---|---|---|
| Uncorrected Fisher's LSD | Diff. | Summary | P Value |
| tTA - Vehicle vs. Tg4510 - Vehicle | −515.5 | ** | 0.0057 |
| tTA - Vehicle vs. tTA - | −265.8 | ns | 0.1309 |
| seltorexant 30 mg/kg | | | |
| Tg4510 - Vehicle vs. Tg4510 - | −178.5 | ns | 0.3316 |
| seltorexant 30 mg/kg | | | |

### Rearing Episodes

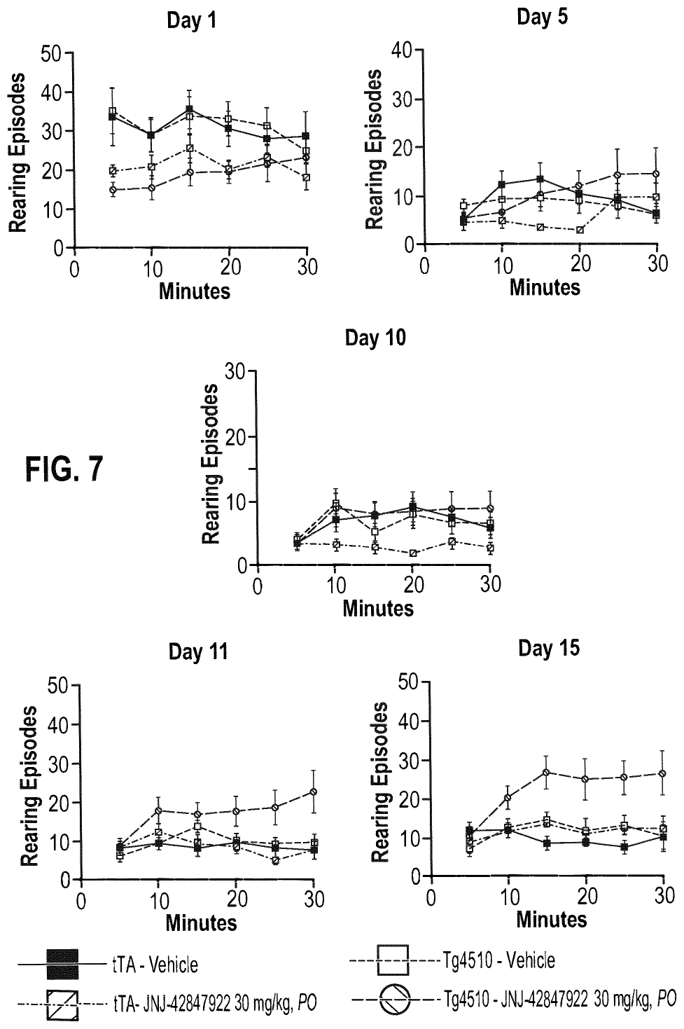

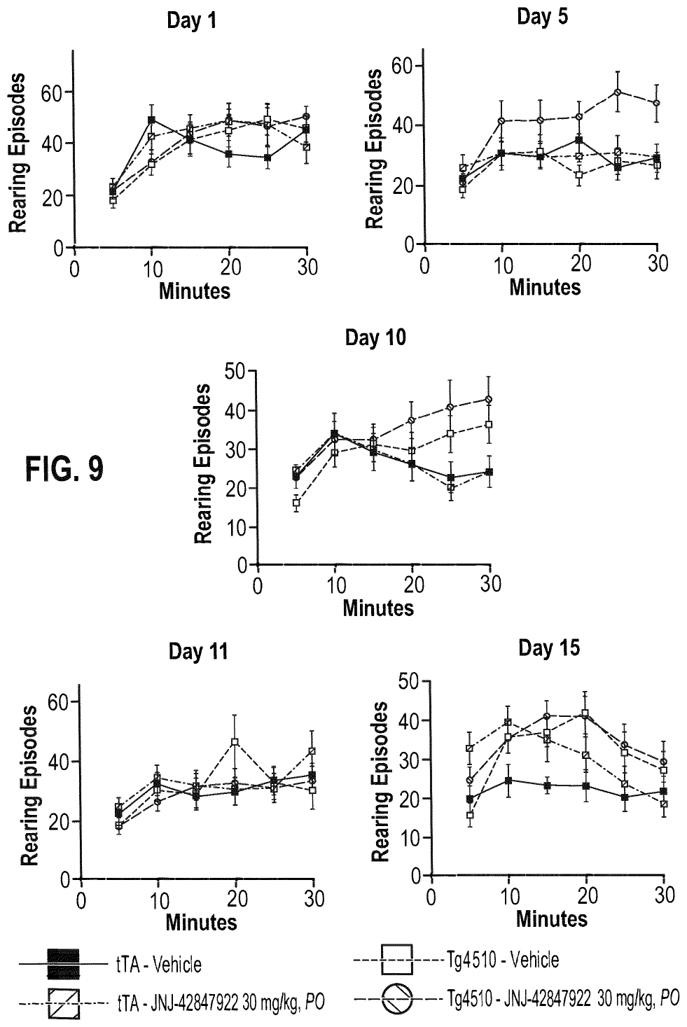

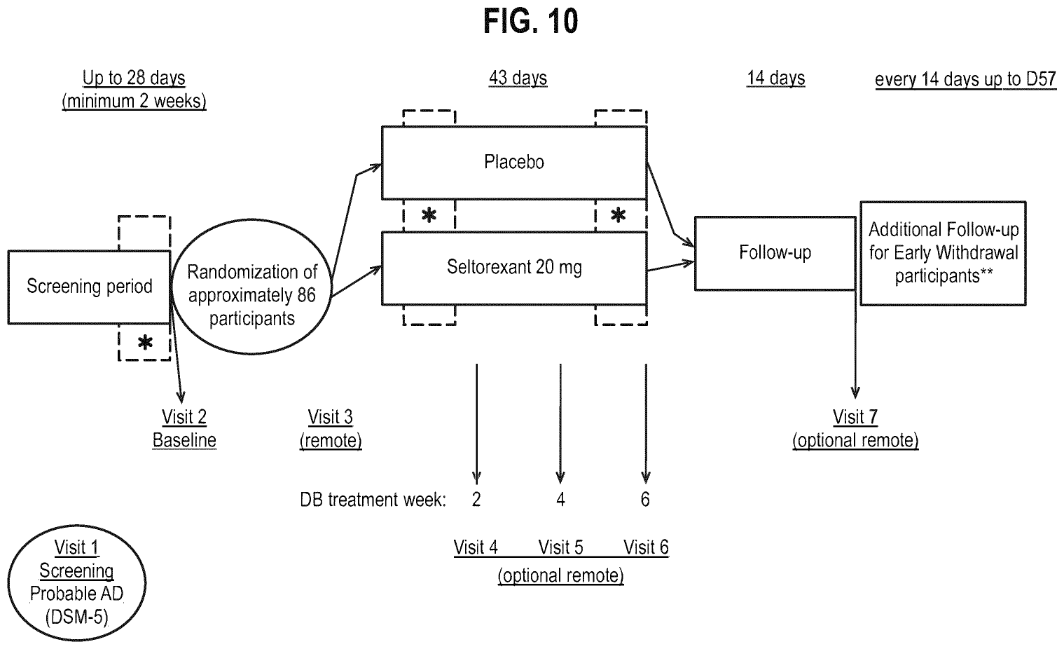

The number of rearing episodes by each group for Days 1, 5, 10, 11, and 15 is shown in FIG. 6 (or FIG. 6A) for the AM test sessions and in FIG. 8 (or FIG. 8A) for the PM test sessions. Time course data are summarized in FIG. 7 (or FIG. 7A) (AM) and FIG. 9 (or FIG. 9A) (PM). One-way ANOVA conducted for each AM test session found a significant effect of Treatment on Days 1, 10, 11, and 15. See, Table 9.

TABLE 9
Rearing Episodes - AM Test Sessions

| | | | | | |
|---|---|---|---|---|---|
| ANOVA table | SS | DF | MS | F (DFn, DFd) | P value |

Day 1

| | | | | | |
|---|---|---|---|---|---|
| Treatment (between columns) | 65861 | 3 | 21954 | F (3, 57) = | P = 0.0116 |
| Residual (within columns) | 311333 | 57 | 5462 | 4.019 | |
| Total | 377194 | 60 | | | |

Day 5

| | | | | | |
|---|---|---|---|---|---|
| Treatment (between columns) | 6401 | 3 | 2134 | F (3, 55) = | P = 0.3904 |
| Residual (within columns) | 114932 | 55 | 2090 | 1.021 | |
| Total | 121333 | 58 | | | |

Day 10

| | | | | | |
|---|---|---|---|---|---|
| Treatment (between columns) | 6997 | 3 | 2332 | F (3, 55) = | P = 0.0485 |
| Residual (within columns) | 45843 | 55 | 833.5 | 2.798 | |
| Total | 52840 | 58 | | | |

Day 11

| | | | | | |
|---|---|---|---|---|---|
| Treatment (between columns) | 27960 | 3 | 9320 | F (3, 56) = | P = 0.0079 |
| Residual (within columns) | 119731 | 56 | 2138 | 4.359 | |
| Total | 147691 | 59 | | | |

Day 15

| | | | | | |
|---|---|---|---|---|---|
| Treatment (between columns) | 55712 | 3 | 18571 | F (3, 56) = | P = 0.0003 |
| Residual (within columns) | 143237 | 56 | 2558 | 7.260 | |
| Total | 198949 | 59 | | | |

Fisher's LSD test on Day 1 AM found that tTA-seltorexant 30 mg/kg treated mice reared significantly less than tTA-Vehicle mice (p<0.05) and that Tg4510-seltorexant 30 mg/kg treated mice reared significantly less than Tg4510-Vehicle mice (p<0.01). See, Table 10.

TABLE 10
Day 1 - AM

| | Mean | | Individual |
|---|---|---|---|
| Uncorrected Fisher's LSD | Diff. | Summary | P Value |
| tTA - Vehicle vs. Tg4510 - Vehicle | −1.854 | ns | 0.9446 |
| tTA - Vehicle vs. tTA - | 57.28 | * | 0.0353 |
| seltorexant 30 mg/kg | | | |
| Tg4510 - Vehicle vs. Tg4510 - | 72.80 | ** | 0.0092 |
| seltorexant 30 mg/kg | | | |

Fisher's LSD test on Day 10 AM found that tTA-seltorexant 30 mg/kg treated mice reared significantly less than tTA-Vehicle mice (p<0.05). See, Table 11.

TABLE 11
Day 10 - AM

| | Mean | | Individual |
|---|---|---|---|
| Uncorrected Fisher's LSD | Diff. | Summary | P Value |
| tTA - Vehicle vs. Tg4510 - Vehicle | 0.9095 | ns | 0.9327 |
| tTA - Vehicle vs. tTA - | 22.80 | * | 0.0349 |
| seltorexant 30 mg/kg | | | |
| Tg4510 - Vehicle vs. Tg4510 - | −6.376 | ns | 0.5547 |
| seltorexant 30 mg/kg | | | |

On Days 11 and 15 AM, Fisher's LSD test found that Tg4510-seltorexant 30 mg/kg treated mice reared significantly more than Tg4510-Vehicle mice (p<0.05-p<0.001). See, Table 12.

TABLE 12

| | | | |
|---|---|---|---|
| | Mean | | Individual |
| Uncorrected Fisher's LSD | Diff. | Summary | P Value |

Day 11 - AM

| | | | |
|---|---|---|---|
| tTA - Vehicle vs. Tg4510 - Vehicle | −6.686 | ns | 0.6987 |
| tTA - Vehicle vs. tTA - | 0.2667 | ns | 0.9875 |
| seltorexant 30 mg/kg | | | |
| Tg4510 - Vehicle vs. Tg4510 - | −43.78 | * | 0.0123 |
| seltorexant 30 mg/kg | | | |

Day 15 - AM

| | | | |
|---|---|---|---|
| tTA - Vehicle vs. Tg4510 - Vehicle | −10.66 | ns | 0.5728 |
| tTA - Vehicle vs. tTA - | −10.20 | ns | 0.5829 |
| seltorexant 30 mg/kg | | | |
| Tg4510 - Vehicle vs. Tg4510 - | −64.45 | *** | 0.0010 |
| seltorexant 30 mg/kg | | | |

One-way ANOVA conducted for each PM test session found a significant effect of Treatment on Day 15. See, Table 13.

TABLE 13
Rearing Episodes - PM Test Sessions

| | | | | | |
|---|---|---|---|---|---|
| ANOVA table | SS | DF | MS | F (DFn, DFd) | P value |

Day 1

| | | | | | |
|---|---|---|---|---|---|
| Treatment (between columns) | 4124 | 3 | 1375 | F (3, 55) = | P = 0.8936 |
| Residual (within columns) | 371661 | 55 | 6757 | 0.2034 | |
| Total | 375785 | 58 | | | |

Day 5

| | | | | | |
|---|---|---|---|---|---|
| Treatment (between columns) | 71585 | 3 | 23862 | F (3, 57) = | P = 0.0566 |
| Residual (within columns) | 511113 | 57 | 8967 | 2.661 | |
| Total | 582698 | 60 | | | |

Day 10

| | | | | | |
|---|---|---|---|---|---|
| Treatment (between columns) | 25682 | 3 | 8561 | F (3, 59) = | P = 0.2826 |
| Residual (within columns) | 388196 | 59 | 6580 | 1.301 | |
| Total | 413878 | 62 | | | |

Day 11

| | | | | | |
|---|---|---|---|---|---|
| Treatment (between columns) | 4159 | 3 | 1386 | F (3, 58) = | P = 0.8828 |
| Residual (within columns) | 367041 | 58 | 6328 | 0.2191 | |
| Total | 371200 | 61 | | | |

Day 15

| | | | | | |
|---|---|---|---|---|---|
| Treatment (between columns) | 42698 | 3 | 14233 | F (3, 57) = | P = 0.0438 |
| Residual (within columns) | 281851 | 57 | 4945 | 2.878 | |
| Total | 324549 | 60 | | | |

Fisher's LSD test on Day 15 PM found that Tg4510-Vehicle mice reared significantly more than tTA-Vehicle mice (p<0.05). See, Table 14.

TABLE 14
Day 14 - PM

| | Mean | | Individual |
|---|---|---|---|
| Uncorrected Fisher's LSD | Diff. | Summary | P Value |
| tTA - Vehicle vs. Tg4510 - Vehicle | −55.80 | * | 0.0339 |
| tTA - Vehicle vs. tTA - | −47.55 | ns | 0.0650 |
| seltorexant 30 mg/kg | | | |
| Tg4510 - Vehicle vs. Tg4510 - | −15.73 | ns | 0.5425 |
| seltorexant 30 mg/kg | | | |

### Summary

The goal of this study was to determine the efficacy of seltorexant (30 mg/kg) on locomotor activity in Tg4510 mice. In terms of distance traveled on the first day of open field testing (Day 1 AM), vehicle-treated Tg4510 animals traveled significantly more compared to vehicle-treated tTA animals. The hyperactivity observed in the Tg4510 group on Day 1 (AM) was significantly attenuated in the presence of seltorexant (30 mg/kg). In contrast, during the evening test on Day 5 (PM), seltorexant (30 mg/kg) increased distance traveled in the Tg4510 group. The hyperactivity observed in the Tg4510-Vehicle group on Day 1 (AM), reappeared on Day 15 (5 days after the completion of dosing).

In terms of rearing episodes, seltorexant (30 mg/kg) reduced rearing in the tTA-Vehicle group during the AM sessions on Day 1 and Day 10. The compound similarly reduced rearing on Day 1 (AM) in the Tg4510 group. Upon completion of dosing, increased rearing episodes were observed in the vehicle treated (Day 15 PM) and seltorexant (30 mg/kg)-treated (Day 11 AM; Day 15 AM) Tg4510 animals.

### EXAMPLE 2—PROPHETIC EXAMPLE OF HUMAN STUDY

This multi-center randomized, placebo-controlled, double-blind study will investigate seltorexant safety, tolerability, and clinical efficacy in approximately 86 participants with probable AD and clinically significant agitation/aggression. Participants will be diagnosed with probable AD (DSM-5), and criteria of a syndrome diagnosis of agitation based on IPA consensus clinical and research definition of agitation in cognitive disorders, have an MMSE total score from 10 to 24 (inclusive), and a CDR of ≥2. Additionally, patients will be characterized by the presence of clinically significant agitation/aggression defined as NPI-12 agitation/aggression (A/A) domain score ≥4 with NPI frequency score ≥2 at entry and baseline with no more than 35% of improvement allowed in NPI-12 A/A score during the Screening period. NPI-12 will be used to enrich the study population for sleep problems (targeting for approximately 50% or more of the participants who should meet NPI-12 sleep domain score of ≥4). Vascular factors associated with the dementia will be assessed with the HIS at baseline. Biomarkers associated with AD will also be assessed at baseline.

Each participant in the study will have a designated study partner who will be available to meet with study staff at each visit. A study partner can be a relative, friend or caretaker indicated by the participant (or legal representative), who is at least 18 years old, who may or may not live with the participant, who has at least 8 hours of contact with the participant each week (e.g., 4 days a week for at least 2 hours per day), and who is available for contact with the study site staff at each visit. Prior to study assessments, study partners will receive oral and written information about the study.

### A. Study Design

This study will consist of 3 phases: a screening phase (up to 28 days), a double-blind treatment phase (43 days), and a post-treatment follow-up phase (14 days after DB treatment phase). Eligible participants will be randomized in a 1:1 ratio after the screening period to receive either placebo or seltorexant 20 mg for 6 consecutive weeks.

### Sequence and Duration of Study Phases/Periods:

The duration of participation in the study for an individual participant (including screening, DB treatment, and follow-up phases) will be approximately 14 weeks. There will be up to 6 clinic visits (Visit 1, 2, 3 [remote], 4, 5 [optional remote], 6, & 7 [optional remote]).

TABLE 15
Schedule of Activities

| | | DB Treatment | | | | |
|---|---|---|---|---|---|---|
| | | | | | | End of DB |
| | | | | | | Treatment/ |
| | | | Remote | | | Early |
| Period | SCR | Baseline | contact^{f} | | | Withdrawal |
| Study Day | D-28 to D-1 (up | D1 | D8 | D15 | D29 | D43 |
| | to 4 weeks prior | | | | | |
| | to | | | | | |
| | randomization)^{p} | | | | | |
| Study Week | | | 1 | 2 | 4 | 6 |
| Visit | 1 | 2 | 3 | 4 | 5 | 6 |
| | | | (remote) | | (optional | |
| | | | | | remote)ⁱ | |
| Visit window | | | ±2 | ±3 | ±3 | ±3 |
| (Days) | | | | | | |
| Study | | | | | | |
| Procedure | | | | | | |

Screening/Administrative

| | | | | | | |
|---|---|---|---|---|---|---|
| Demographics | X | | | | | |
| Probable AD | X | | | | | |
| diagnosis- | | | | | | |
| DSM-5 and | | | | | | |
| agitation | | | | | | |
| confirmed with | | | | | | |
| IPA criteria | | | | | | |
| CDR | X | | | | | |
| MMSE | X | | | | | X |
| Medical | X | | | | | |
| history | | | | | | |
| Family and | X | | | | | |
| disease history | | | | | | |
| Inclusion/ | X | X | | | | |
| exclusion | | | | | | |
| criteria^{b} | | | | | | |
| Study | X | X | | | | |
| restrictions | | | | | | |
| Urine drug | X | X | | | | X |
| screen | | | | | | |
| Alcohol breath | X | X | | X | | X |
| test | | | | | | |
| Preplanned | X | | | | | |
| surgery/ | | | | | | |
| procedure(s) | | | | | | |
| Brief | X^{e} | X^{e} | | | | |
| psychosocial | | | | | | |
| therapy | | | | | | |
| HIS | | X | | | | |
| Query for AEs, | | | X^{f} | | X^{f} | |
| dosing | | | | | | |
| compliance | | | | | | |
| and tolerance, | | | | | | |
| and medication | | | | | | |
| diary | | | | | | |
| completion | | | | | | |

Study Intervention Administration

| Randomization | X^{g} | | | | |
|---|---|---|---|---|---|
| Study | X | Once a day nightly | | | |
| intervention | | | | | |
| intakeⁿ | | | | | |
| Dispensing | X | | X | X | |
| study drug | | | | | |
| Medication | | X | X | X | X |
| diary review | | | | | |
| Study drug | | X^{l} | X | X^{l} | X |
| accountability | | | | | |

Efficacy Assessments

| | | | | | |
|---|---|---|---|---|---|
| NPI-12 | X | X | X | X | X |
| NPI-C | | X | X | X | X |
| SDI | X | X | X | X | X |
| CMAI-C | | X | X | | X |

Safety Assessments

| | | | | | |
|---|---|---|---|---|---|
| Physical | X | X | | | X |
| examination | | | | | |
| Neurological | X | X | | | X |
| examination | | | | | |
| Height | X | | | | |
| Weight | X | X | | | X |
| Vital signs^{c} | X | X | X | | X |
| 12-lead ECG^{c} | X | X | X | | X |
| C-SSRS | X | X | X | X | X |
| ADAS-Cog | | X | X | | X |
| PWC-20 | | | | | X |

Clinical Laboratory Tests

| | | | |
|---|---|---|---|
| Hematology, | X | X | X |
| chemistry^{d} | | | |
| Urinalysis | X | X | X |

Clinical Pharmacology Assessments

| | | |
|---|---|---|
| Blood sample | X | X |
| collection for | | |
| PK^{j} | | |
| Alpha-1-acid | X | X |
| glycoprotein | | |

Biomarkers

| | | |
|---|---|---|
| Blood sample | X | X |
| collection | | |

Genetic and Pharmacogenomic (DNA) Assessments

| Blood sample | | X | | |
|---|---|---|---|---|
| collection^{m} | | | | |
| Digital | | | | |
| Actigraphy | X | X | X | |
| device | | | | |
| instructions | | | | |
| Actigraphy | | X | X | X |
| device | | | | |
| performance | | | | |
| check | | | | |
| Actigraphy | Continuous^{h} | | | |
| device | | | | |
| measurements | | | | |
| Off-body | Continuous^{q} | | | |
| sensors (in a | | | | |
| subset of the | | | | |
| participants) | | | | |

Ongoing Participant Review

| Concomitant | Continuous |
|---|---|
| therapy | |

| | |
|---|---|
| Adverse events | Continuous |

Schedule of Activities

| | | Additional FU visit for EW |
|---|---|---|
| Period | Follow-Up | of study drug^{k} |
| Study Day | D57 (2 weeks after | Every 2 weeks up to Day 57 |
| | completing the | |
| | treatment period) | |
| Study Week | 8 | |
| Visit | 7 (optional remote)ⁱ | [optional remote] |
| Visit window (Days) | +/−3 | |
| Study Procedure | | |

Screening/Administrative

Demographics
Probable AD diagnosis-DSM-5 and
agitation confirmed with IPA criteria
CDR
MMSE
Medical history
Family and disease history
Inclusion/exclusion criteria^{b}
Study restrictions
Urine drug screen
Alcohol breath test
Preplanned surgery/procedure(s)
Brief psychosocial therapy
HIS
Query for AEs, dosing compliance
and tolerance, and medication diary
completion

Study Intervention Administration

Randomization
Study intervention intakeⁿ
Dispensing study drug
Medication diary review
Study drug accountability

Efficacy Assessments

| | | |
|---|---|---|
| NPI-12 | X | X |
| NPI-C | X | X |
| SDI | X | X |
| CMAI-C | | |

Safety Assessments

Physical examination
Neurological examination
Height
Weight

| | | |
|---|---|---|
| Vital signs^{c} | X^{o} | X |
| 12-lead ECG^{c} | | |
| C-SSRS | X | X |
| ADAS-Cog | | |
| PWC-20 | X | |

Clinical Laboratory Tests

Hematology, chemistry^{d}
Urinalysis

Clinical Pharmacology Assessments

Blood sample collection for PK^{j}
Alpha-1-acid glycoprotein

Biomarkers

Blood sample collection

Genetic and Pharmacogenomic (DNA) Assessments

Blood sample collection^{m}

Digital

Actigraphy device instructions
Actigraphy device performance check
Actigraphy device measurements
Off-body sensors (in a subset of the
participants)

Ongoing Participant Review

| | |
|---|---|
| Concomitant therapy | Continuous |
| Adverse events | Continuous |

^{a}Must be signed before first study-related activity.
^{b}Minimum criteria for the availability of documentation supporting the eligibility criteria are described in Source Documentation in Appendix 3, Regulatory, Ethical, and Study Oversight Considerations. Check clinical status again before first dose of study medication.
^{c}Supine vital signs should be measured after 5 minutes of rest in a supine position. If blood sampling or vital sign measurement is scheduled for the same time point as ECG recording, the procedures should be performed in the following order: ECG(s), vital signs, blood draws.
^{d}If possible, participants should be fasted (i.e., no food or beverages [except water]) for at least 8 hours before blood is drawn for clinical chemistry and hematology (with the possible exception of screening laboratory tests).
^{e}Participants and study partners will be offered brief psychosocial therapy during screening. A number of sessions will be scheduled during the screening period and should be completed at Visit 2 (Baseline).
^{f}For the remote contact at Visit 3, approximately 7 days after Visit 2 and at Visit 5 if done remotely, participants and their study partners will be contacted remotely (i.e., by telephone or videoconference) and queried for AEs, overall seltorexant dosing compliance and tolerance, any changes in concomitant therapies, and medication diary completion.
^{g}Eligible participants who successfully complete Visit 2 will be randomized and receive the study medication to take home until the next visit of the double-blind treatment period, including instructions and a medication diary.
^{h}Participants will wear the actigraphy device continuously throughout the study upon receipt. At Visit 6, participants and study partners will return the actigraphy device.
ⁱVisits 5 and 7 may be done remotely (e.g., tele- or videoconference; optional) or at the research site.
^{j}For all participants, 1 PK sample will be collected in the morning on Day 15 between approximately 8 and 14 hours after dosing at night on Day 14 and 1 PK sample will be collected in the morning on Day 43, between approximately 8 and 14 hours after dosing at night on Day 42. Study drug dosing and last mealtime on the day before each PK sample collection will be accurately recorded by exact dosing date and time and last meal date and time by the participant and/or study partner in the participant medication diary. In addition, blood samples will be collected for determination of plasma concentrations of seltorexant, its M12 metabolite, and alpha-1-acid glycoprotein in participants who discontinue study drug for an AE, have an AESI, or have an SAE if the sample can be obtained within 15 hours of the last study drug administration.
^{k}Participants who discontinue treatment during the double-blind phase prior to Day 35 and do not withdraw consent for further assessments, will have End-of-DB Treatment visit and Follow-up visit, and will be encouraged to have additional Follow-up visits with assessments every 2 weeks until Day 57.
^{l}For Day 8 remote contact and other remote visits, the site will ask about adherence to study drug, but formal drug accountability (pill counts) will not be done.
^{m}The genetic and pharmacogenomic (DNA) sample should be collected at the specified time point, however, if necessary, it may be collected at a later time point without constituting a protocol deviation.
ⁿStudy intervention intake ends on the night of Day 42.
^{o}Vital signs collection will be done only if an in person site visit is conducted.
^{p}An extension of up to 2 weeks of the screening phase may be allowed (e.g., if needed to confirm eligibility criteria, taper off a sleep medication or for scheduling difficulties). The minimal screening duration should be 2 weeks to allow the proper conduct of the brief psychosocial therapy.
^{q}Off-body sensors will be used in a subset of participants. Participants or their study partner may opt-out of using the off-body sensor. The sensor(s) will be placed in the participants home and be used from screening to end of treatment.

### Screening Period (Minimum 2 Weeks and Up to 28-Days Before the Double-Blind Treatment Period)

### Visit 1:

During screening, participants will be assessed for being able to meet the study inclusion criteria and if they meet any exclusion criteria. Diagnosis will be confirmed based on DMS-5 criteria for AD, and criteria of a syndrome diagnosis of agitation based on IPA consensus clinical and research definition of agitation in cognitive disorders, severity of dementia will be assessed, as well as cognitive functioning, and BPSD symptoms including agitation and aggression. Medical, family, and disease history will be obtained, including prior and concomitant therapies, and safety evaluations (e.g., physical examination, neurological examination, vital signs, ECG, C-SSRS, urine drug screen, alcohol breath test, and clinical laboratory tests) will be performed to assess eligibility.

Participants and study partners who successfully completed the other Visit 1 assessments and meet study participation requirements will be offered brief psychosocial therapy. The first session should occur in person with the study partner during screening Visit 1, and subsequent sessions will be scheduled during the screening period and should be completed at Visit 2 (Baseline). Additionally, participants and study partners will be instructed in the use of the actigraphy device used for at-home recording of activity patterns and sleep patterns. Participants will wear the actigraphy device continuously throughout the study screening and double-blind periods when tolerated. Participants may be included in the study even if they are not able or willing to wear the actigraphy device.

If implemented, the off-body sensors will be placed in the participants home at Screening (subset of the participants who agreed for use of the device at Screening), and the data should be collected for minimum 2 weeks before randomization and until End of DB Treatment.

Prohibited medications will be tapered and discontinued prior to the start of the DB treatment phase as described herein. Treatment of stable medical conditions is allowed as defined in the concomitant medications section.

Adverse events will be collected until the completion of the last study procedure on the final follow-up visit.

An extension of up to 2 weeks of the screening phase may be allowed (e.g., if needed to confirm eligibility criteria, taper off a sleep medication or for scheduling difficulties). The minimal screening duration should be 2 weeks to allow the proper conduct of the brief psychosocial therapy.

### Double-Blind Treatment Period

Eligible participants and study partners will return to the clinic to start the double-blind treatment period within 28 days after start of the screening (Visit 1).

### Visit 2:

Visit 2 will be made on Day 1 (baseline) of study Week 0. Study restrictions and any changes in inclusion/exclusion criteria will be reviewed, and an alcohol breath test and urine drug screen performed. Other assessments will be completed as per Table 15 at this and subsequent visits.

Upon completion of Visit 2 assessments, eligible participants will be randomized and receive the study medication to take home for the double-blind treatment period, including instructions and a medication diary. Participants and study partners will be reminded about the use of the actigraphy device.

### Visit 3

Approximately 7 days after Visit 2 (baseline), participants will be contacted remotely (i.e., by telephone or videoconference) and queried for AEs that may have occurred between the last visit and remote follow up, and overall study drug dosing compliance and tolerance, and medication diary completion.

### Visit 4

Visit 4 will take place at the end of Week 2 of the double-blind treatment period.

Upon completion of Visit 4 assessments, participants and study partners will be reminded about the use of the actigraphy device.

### Visit 5

Visit 5 will be a remote visit (may be done in person if needed) at the end of week 4 of the double-blind treatment period.

### Visit 6

Visit 6 will be at the end of week 6 of the double-blind treatment period or at the time of early withdrawal from study treatment. At Visit 6, participants and study partners will return the actigraphy device.

All participants who discontinue study drug in the DB treatment phase, will have an Early Withdrawal visit (Visit 6 in Table 15) and a Follow-up visit (Visit 7 in Table 15). Participants who discontinue study drug prior to Day 35 are encouraged to continue after the Follow-up visit (Visit 7 in Table 15) with additional follow-up visits every 2 weeks per Table 15 until Day 57.

Efficacy and safety assessments, sparse PK sampling, biomarkers sampling, and other study procedures will be performed at each visit per Table 15. If a participant or study partner withdraws from the study drug, the End-of-DB/Early Withdrawal visit assessments (including NPI-C/12) will be performed per Table 15, preferably on the day after the last dose or as soon as the participant and study partner are available for the assessment.

Entry to the follow-up phase should not be conducted in participants or study partners who withdraw consent for further study assessments; however, they should complete the End-of-DB/EW visit if they are willing to do so.

### Follow-Up Period-Post-Treatment Follow-up period (14 Days After Completion of the Double-Blind Treatment Period)

Visit 7 (Follow-up Visit) which may be done remotely or in person.

Participants who received at least 1 dose of study drug, except those who withdrew consent for the study or who are lost to follow-up, will complete follow-up assessments per Table 15 approximately 14 days after the last dose of study drug.

For participants who have completed the study (at the end of DB treatment phase), this follow-up visit is the last visit. All participants who discontinue study drug in the DB treatment phase, will have an End of DB/Early Withdrawal visit (Visit 6 in Table 15) and a Follow-up visit (Visit 7 in Table 15). Participants who discontinue study drug prior to Day 35 are encouraged to continue after the Follow-up visit (Visit 7 in Table 15) with additional Follow-up visits, every 2 weeks per Table 15 until Day 57.

### Blinding, Control, Study Phase/Periods, Intervention Groups

Treatment assignment will be blinded to reduce potential expectation bias during evaluation of clinical endpoints as well as AEs.

A placebo control will be used to establish the frequency and magnitude of changes in clinical endpoints, both efficacy and safety, that may occur in the absence of active treatment.

Randomization will be used to minimize bias in the assignment of participants to treatment groups, to increase the likelihood that known and unknown participant attributes (e.g., demographic and baseline characteristics) are evenly balanced across treatment groups, and to enhance the validity of statistical comparisons across treatment groups.

Brief psychosocial therapy during screening will be offered in order to reduce non-specific effects of increased attention/inclusion in the trial.

A 14-day follow-up period off treatment will be used to assess short-term rebound effect on behavioral and psychological symptoms of dementia as well as withdrawal effect after discontinuing seltorexant.

### DNA and Biomarker Collection

Pharmacogenomic research may help to explain interindividual variability in clinical outcomes and may help to identify population subgroups that respond differently to an intervention. The goal of the pharmacogenomic component is to collect DNA to allow the identification of genetic factors that may influence the PK, PD, efficacy, safety, or tolerability of seltorexant. In this regard, metabolism of seltorexant may be influenced by genetic variation in CYP2C9 and CYP3A4. An additional goal of the pharmacogenomic component is to assess APOE genotype.

APOE is involved in several key Aβ-mediated processes associated with AD, including the distribution, clearance and/or metabolism of Aβ, altered lipid-binding properties leading to an augmentation of Aβ-mediated toxicity (e.g., disruption of lysosomal storage), and formation of aggregates that may promote Aβ nucleation and plaque formation. The APOE ε4 allele is considered the most significant genetic risk factor for AD and is associated with a decreased age of onset of the disease. Together with other AD blood-based biomarkers (p217+tau, Aβ42/40 levels and ratio) and other exploratory biomarkers of neurodegeneration (such as NfL), APOE genotype may help to explain interindividual variability in clinical outcomes or define subgroups of dementia patients that may respond differentially to seltorexant treatment. DNA and biomarker samples may be used to help address emerging issues and to enable the development of safer, more effective, and ultimately individualized therapies.

### End of Study Definition

A participant will be considered to have completed the DB treatment phase if he or she has completed the Day 43 visit of the DB treatment phase and has not discontinued study drug early during DB phase. A participant will be considered to have completed the follow-up phase if he or she has completed assessments at all follow-up visits.

The end of study is considered as the last study assessment shown in Table 15, completed for the last participant in the study.

### B. Objectives and Endpoints

| | |
|---|---|
| Objectives | Endpoints |

Primary

| | |
|---|---|
| To investigate the effect of seltorexant versus | Change from baseline to Day 43 |
| placebo on the sum of agitation and aggression | on NPI-C A + A scores. |
| domain scores (A + A) of the NPI-C in | |
| participants with probable AD with clinically | |
| significant agitation/aggression. | |

Secondary - Efficacy

To investigate the effect of seltorexant versus placebo on the following in
participants with probable AD with clinically significant agitation/aggression:

| | |
|---|---|
| CMAI-C total score | Change from baseline to Day 43 |
| | on the total CMAI-C score. |
| Sleep symptoms as assessed by the SDI. | Change from baseline to Day 43 |
| | on SDI total score. |

Secondary - Pharmacokinetics

| | |
|---|---|
| To investigate the plasma PK of seltorexant in | Plasma seltorexant |
| participants with probable AD with clinically | concentrations at Day 15 and |
| significant agitation/aggression. | Day 43. |

Secondary - Safety

To investigate the effect of seltorexant versus placebo on the following in
participants with probable AD with clinically significant agitation/aggression:

| | |
|---|---|
| Safety and tolerability of seltorexant | Change from baseline in VS, |
| | Clinical labs (chemistry, |
| | hematology, urinalysis), ECG, |
| | CSSRS, and TEAEs including |
| | AESIs. |
| Cognitive symptoms | Change from baseline to Day 43 |
| | on ADAS-Cog-14 composite |
| | cognitive score and from |
| | screening to Day 43 on the |
| | MMSE total score. |

Exploratory - Digital

To investigate the effect of seltorexant versus placebo on the following in
participants with probable AD with clinically significant agitation/aggression:

| | |
|---|---|
| Sleep and activity metrics using an actigraphy | Change from baseline on sleep |
| device. | and activity metrics. |
| Sleep and pacing using an off-body sensor in a | Change from baseline on sleep |
| subset of participants | and pacing measurements |

Exploratory - Biomarkers

| | |
|---|---|
| To investigate the effect of seltorexant versus | Change from baseline in levels |
| placebo in participants with probable AD with | of blood biomarkers of AD |
| clinically significant agitation/aggression on | |
| blood biomarkers including but not limited to: | |
| biomarkers of AD, e.g., p217 + tau, | |
| Aβ_{1-42/40} levels and ratio, NfL, and | |
| other exploratory biomarkers | |

Other Exploratory

To investigate the effect of seltorexant versus placebo on the following in
participants with probable AD with clinically significant agitation/aggression:

| | |
|---|---|
| Variables derived from the NPI-12/NPI-C | Change from baseline over time |
| rating: | on the specified NPI-12/NPI-C |
| The sum of the NPI-C delusion and | variables. |
| hallucination domain scores (NPI-C | |
| D + H) | |
| Individual NPI-C domain scores for agitation, | |
| aggression, dysphoria, delusions, and | |
| hallucinations | |
| NPI-12 total score | |
| NPI-12 Caregiver Distress total score | |
| All NPI-12 individual item scores | |
| SDI Caregiver Distress score | Change from baseline over time |
| | in SDI caregiver distress |
| Short-term rebound effects on behavioral and | Change in PWC-20 from End of |
| psychological symptoms of dementia as well as | DB Treatment/EW to first |
| withdrawal symptoms | follow up visit. |
| | Change in NPI-12, NPI-C and |
| | SDI scores from End of DB |
| | Treatment/EW to first follow up |
| | visit. |
| CMAI-C factor scores | Change from baseline over time |
| | on CMAI-C factor scores. |
| Explore the association between treatment effect | Correlation between change in NPI- |
| of seltorexant in sleep and treatment effect in | C A + A and change in SDI total |
| agitation/aggression | score, and mediation effect on |
| | change in NPI-C A + A through |
| | change in SDI total score |

### C. Study Population

Approximately 86 participants with probable AD and clinically significant agitation/aggression will be enrolled in this study according to the inclusion and exclusion criteria specific for this study. Screening for eligible participants will be performed within 28 days before administration of the study intervention.

### 1. Inclusion Criteria

Each potential participant must satisfy all of the following criteria to be enrolled in the study:

| | |
|---|---|
| 1. | Age: 55 to 85 years of age, inclusive. |

Type of Participant and Disease Characteristic

| | |
|---|---|
| 2. | Participant has received a diagnosis of probable Alzheimer's disease (DSM-5) with the |
| | following characteristics at Screening: |
| | CDR global score ≥2 |
| | MMSE total score of 10 to 24 (inclusive) |
| 3. | Participant meets the criteria of a syndrome diagnosis of agitation based on IPA |
| | consensus clinical and research definition of agitation in cognitive disorders for at least |
| | 2 weeks before Screening (see Table 16 for the criteria) |
| 4. | Participant meets the criteria of NPI-12 A/A domain score ≥4 with frequency score |
| | ≥2 at Screening and Baseline with no more than 35% of improvement in NPI-12 A/A |
| | domain score from the Screening to Baseline assessments |
| 5. | Participant is medically stable on the basis of clinical laboratory tests performed at |
| | screening. |
| 6. | Participant must be medically stable on the basis of the following: physical |
| | examination, neurological examination, vital signs (including blood pressure), and 12- |
| | lead ECG performed at screening and baseline. If there are any abnormalities that are |
| | not specified in the inclusion and exclusion criteria, their clinical significance must be |
| | determined. |
| 7. | Participant is considered eligible according to the following Covid-19 screening |
| | criteria: |
| | Has no history of possible or confirmed Covid-19 at screening and within 14 |
| | days prior to screening. Possible or confirmed COVID-19 diagnosis may be |
| | based on: |
| | Symptomatology—at least 1 of the following main symptoms: cough, |
| | dyspnea, thoracic pain, acute anosmia or dysgeusia without clear |
| | etiology OR have at least 2 of the following symptoms: fever, muscle |
| | pain, fatigue, rhinitis, sore throat, headache, anorexia, watery diarrhea |
| | without clear etiology, acute mental confusion, acute falls without clear |
| | etiology OR worsening of chronic respiratory infections (COPD, |
| | asthma, chronic cough . . . ) |
| | Radiology—suggestive clinical presentation AND compatible thorax |
| | CT, even if laboratory Covid-19 antigen test is negative |
| | Laboratory test—positive Covid-19 antigen test |
| | Signs or symptoms suggestive of active Covid-19 upon medical history |
| | and/or physical examination. |
| | Recent (within 14 days) close contact with a person with possible or |
| | confirmed Covid-19. |
| 8. | Weight: BMI within the range 18-40 kg/m² (inclusive). |

Sex and Contraceptive/Barrier Requirements

| | |
|---|---|
| 9. | Female participants must be postmenopausal before study entry (amenorrhea for at least |
| | 12 months). |
| 10. | Male participant: |
| | who is heterosexually active with a woman of childbearing potential must agree |
| | to use a double-barrier (a combination of male condom with either cap, |
| | diaphragm, or sponge with spermicide) method of birth control and his female |
| | partner must use a highly effective method of contraception. |
| | must not to donate sperm during the study and for 3 months after receiving the |
| | last dose of study agent |
| | who is sexually active with a woman who is pregnant must use a condom. |
| | must not plan to father a child while enrolled in this study or within 3 months |
| | after the last dose of study intervention |

TABLE 16
IPA consensus clinical and research definition of agitation in cognitive
disorders

| 1. | The patient meets criteria for a cognitive impairment or dementia syndrome (e.g., AD, | |
|---|---|---|
| | FTD, DLB, vascular dementia, other dementias, a pre-dementia cognitive impairment | |
| | syndrome such as mild cognitive impairment or other cognitive disorder). | |
| 2. | The patient exhibits at least one of the following behaviors that are associated with | |
| | observed or inferred evidence of emotional distress (e.g. rapid changes in mood, | |
| | irritability, outbursts). The behavior has been persistent or frequently recurrent for a | |
| | minimum of two weeks and represents a change from the patient's usual behavior. | |
| | a) | Excessive motor activity (examples include: pacing, rocking, gesturing, pointing |
| | | fingers, restlessness, performing repetitious mannerisms). |
| | b) | Verbal aggression (e.g., yelling, speaking in an excessively loud voice, using |
| | | profanity, screaming, shouting). |
| | c) | Physical aggression (e.g., grabbing, shoving, pushing, resisting, hitting others, |
| | | kicking objects or people, scratching, biting, throwing objects, hitting self, slamming |
| | | doors, tearing things, and destroying property). |
| 3. | Behaviors are severe enough to produce excess disability, which in the clinician's | |
| | opinion is beyond that due to the cognitive impairment and including at least one of the | |
| | following: | |
| | a) | Significant impairment in interpersonal relationships. |
| | b) | Significant impairment in other aspects of social functioning. |
| | c) | Significant impairment in ability to perform or participate in daily living activities. |

| | |
|---|---|
| 4. | While co-morbid conditions may be present, the agitation is not attributable solely to |
| | another psychiatric disorder, suboptimal care conditions, medical condition, or the |
| | physiological effects of a substance. |

TABLE 17
Benzodiazepine Equivalence Table

| | Approximately |
|---|---|
| | Equivalent |
| Benzodiazepines | Oral dosages (mg) |
| Alprazolam (Xanax, Xanor, Tafil) | 0.5 |
| Bromazepam (Lexotan, Lexomil) | 5-6 |
| Chlordiazepoxide (Librium) | 25 |
| Clobazam (Frisium) | 20 |
| Clonazepam (Klonopin, Rivotril) | 0.5 |
| Clorazepate (Tranxene) | 15 |
| Diazepam (Valium) | 10 |
| Estazolam (ProSom, Nuctalon) | 1-2 |
| Flunitrazepam (Rohypnol) | 1 |
| Flurazepam (Dalmane) | 15-30 |
| Halazepam (Paxipam) | 20 |
| Ketazolam (Anxon) | 15-30 |
| Loprazolam (Dormonoct) | 1-2 |
| Lorazepam (Ativan, Temesta, Tavor) | 1 |
| Lormetazepam (Noctamid) | 1-2 |
| Medazepam (Nobrium) | 10 |
| Nitrazepam (Mogadon) | 10 |
| Nordazepam (Nordaz, Calmday) | 10 |
| Oxazepam (Serax, Serenid, Serepax, Seresta) | 20 |
| Prazepam (Centrax, Lysanxia) | 10-20 |
| Quazepam (Doral) | 20 |
| Temazepam (Restoril, Normison, Euhypnos) | 20 |
| Triazolam (Halcion) | 0.5 |

### 2. Exclusion Criteria

Any potential participant who meets any of the following criteria will be excluded from participating in the study:

Medical Conditions

| | |
|---|---|
| 1. | Participant fulfils diagnostic criteria for non-Alzheimer's Dementia: e.g., FTD, DLBD, |
| | and post-stroke dementia, based on clinical history. (Participants may be included with |
| | mixed AD/vascular dementia) |
| 2. | Participant has a diagnosed sleep disorder other than insomnia disorder or hypersomnia |
| | such as obstructive sleep apnea or periodic limb movement disorder, REM sleep |
| | behavior disorder based on medical history. |
| 3. | Participant has a neurological, psychiatric or medical condition associated with a long- |
| | term risk of significant cognitive impairment or dementia including but not limited to |
| | pre-manifest Huntington's disease, multiple sclerosis, Parkinson's disease, Down |
| | syndrome, or major psychiatric disorders such as history or current diagnosis of |
| | schizophrenia, schizoaffective or bipolar disorder. |
| 4. | Participants with a history of delirium within 30 days prior to or during screening. |
| 5. | Participant with a cause of agitation that is not secondary to dementia (such as pain) or |
| | significant history of aggression prior to dementia. |
| 6. | Has a history of narcolepsy or seizures (except childhood seizures). |
| 7. | Participant has evidence of intracranial pathology which may affect cognition including |
| | but not limited to brain tumours (benign or malignant), aneurysm or AV malformations, |
| | territorial stroke (excluding smaller watershed strokes), recent haemorrhage |
| | (parenchymal or subdural), or obstructive hydrocephalus. Research participants with an |
| | MRI scan demonstrating markers of small vessel disease (e.g., white matter changes or |
| | lacunar infarcts) judged to be clinically insignificant, or microbleeds are allowed |
| 8. | Participant has clinically significant hepatic disease as defined by: |
| | ≥2x ULN increase of AST or ALT at screening (one retest is permitted) |
| | significant liver disease including cirrhosis, ascites, active hepatitis etc (fatty |
| | liver disease and Gilbert's syndrome will be allowed as long as it does not meet |
| | above criteria). |
| 9. | Participants with clinically significant B12 or folate abnormalities at screening |
| 10. | Has a recent (last 3 months) history of, or current signs and symptoms of, |
| | severe renal insufficiency (CrCl <30 mL/min); |
| | clinically significant or unstable cardiovascular, respiratory, gastrointestinal, |
| | neurologic, hematologic, rheumatologic, immunologic or endocrine disorders. |
| | uncontrolled Type 1 or Type 2 diabetes mellitus. |
| | Participants with Type 1 or Type 2 diabetes mellitus who are controlled (hemoglobin |
| | Al_{C} ≤8.5% and glucose ≤150 mg/dL at screening) may be eligible to participate if |
| | otherwise medically stable, and if on a stable regimen of glucose-lowering medications |
| | for at least 2 months prior to screening. |
| 11. | Has clinically significant ECG abnormalities at screening or Day 1 prior to |
| | randomization that may jeopardize the participants' safety or the integrity of the study |
| | defined as: |
| | During screening and/or Day 1, a QTcF: ≥450 msec (males); ≥470 msec (females). |
| | If the QTcF is prolonged on the initial ECG at a given time point, the average QTcF |
| | of 3 ECGs, recorded 4 minutes apart, must not be ≥450 msec for males and ≥470 |
| | msec for females. |
| | Evidence of 2nd and 3rd degree atrioventricular block. |
| | Features of new ischemia |
| | Other clinically important arrhythmia or cardiac abnormalities |
| 12. | Participant has a clinically significant acute illness within 7 days prior to study |
| | intervention administration |
| 13. | Participant has any cancer or history of cancer (excluding cutaneous basal or squamous |
| | cell cancer resolved by excision), unless in remission or considered stable on hormone |
| | therapy for at least 6 months before screening. |
| 14. | Participant has current signs/symptoms of hypothyroidism or hyperthyroidism. For |
| | participants with a history of thyroid disease and for participants who, regardless of |
| | thyroid history have the TSH value out of range, a FT4 test will be conducted. If the |
| | FT4 value is abnormal and considered to be clinically significant (after discussion with |
| | the sponsor's medical monitor or designee) the participant is not eligible. |
| | Participants with a pre-existing history of thyroid disease/disorder who are treated with |
| | thyroid hormones need to be on a stable dosage for 3 months prior to the start of the |
| | screening phase in order to be eligible for participation. |
| 15. | Participant has a clinically significant condition which may deem the participant's |
| | participation in an investigational trial unsafe, e.g., symptomatic cardiovascular disease |
| | (including re-vascularisation procedures within the previous year), severe renal or |
| | hepatic failure, any clinically relevant abnormalities in blood parameters included in |
| | local routine assessments, severe loss of vision, hearing or communicative ability, |
| | conditions preventing co-operation or completing the required assessments in the trial |
| 16. | Participant has known allergies, hypersensitivity, or intolerance to seltorexant or its |
| | excipients |
| 17. | Participant has a history of drug or alcohol use disorder according to Diagnostic and |
| | Statistical Manual of Mental Disorders (latest edition) criteria within 6 months before |
| | screening or positive test result(s) for alcohol and/or drugs of abuse [opiates (including |
| | methadone), cocaine, amphetamines, methamphetamines, cannabinoids, barbiturates, |
| | MDMA] at screening or baseline |
| 18. | Participant has a current or recent history of homicidal ideation or serious suicidal |
| | ideation within the past 6 months, corresponding to a score of 4 (active suicidal ideation |
| | with some intent to act, without specific plan) or 5 (active suicidal ideation with specific |
| | plan and intent) for ideation on the C-SSRS, or a history of suicidal behavior within the |
| | past year, as validated by the C-SSRS at screening or baseline. |
| 19. | Has Cushing's Disease, Addison's Disease, or other evidence of significant medical |
| | disorders of the HPA axis. |
| 20. | Any condition for which participation would not be in the best interest of the participant |
| | (e.g., compromise the well-being) or that could prevent, limit, or confound the protocol- |
| | specified assessments) |
| 21. | Participant has any other factors that could contraindicate the participation of the |
| | research participant into this trial |

Prior/Concomitant Therapy

| | |
|---|---|
| 22. | Participants who are not stable on concomitant medications or take prohibited |
| | medications |
| 23. | Participant received an investigational intervention (including investigational vaccines) |
| | or used an invasive investigational medical device within 60 days (or 5 half-lives, |
| | whichever is longest) before the planned first dose of study intervention or is currently |
| | enrolled in an investigational study |
| 24. | Has taken a strong inhibitor of CYP3A4 or CYP2C9 or moderate/strong inducer of |
| | CYP3A4 or CYP2C9 or a dual inhibitor/inducer of CYP3A4 and CYP2C9 within 14 |
| | days before the first study drug administration on Day 1 or will require treatment during |
| | the study. |

Other Exclusions

| | |
|---|---|
| 26. | Participant is unable to swallow oral medication without requiring crushing. |
| 28. | Participant has had major surgery, (e.g., requiring general anesthesia) within 8 weeks |
| | before screening, or will not have fully recovered from surgery, or has surgery planned |
| | during the time the participant is expected to participate in the study or within 4 weeks |
| | after the last dose of study intervention administration. |
| 29. | Any condition for which participation would not be in the best interest of the participant |
| | (e.g., compromise the well-being) or that could prevent, limit, or confound the protocol- |
| | specified assessments |

### 3. Lifestyle Considerations

Potential participants are recommended to follow these lifestyle restrictions during the study:
(i). Participant drinks, on average, no more than 5 cups of tea/coffee/cocoa/caffeinated cola per day.
(ii). Abstain from alcohol 24 hours prior to all scheduled visits. On average, 1 or fewer drinks of alcohol per day is allowed.
(iii). Avoidance of blood donation during the study and for at least 90 days after the last visit
(iv). Participants should be cautioned not to drive a car or operate machinery or engage in any potentially hazardous activities if they have had insufficient sleep following administration of the study drug or at any time during the study if the participant feels that his or her baseline competency is impaired, such as feeling sedated.

### Participant Identification, Enrollment, and Screening Logs

Each participant must have a study partner in order to be eligible for the trial.

For purposes of this trial, the subject's study partner is defined as the person who is at least 18 years old, who may or may not live with the participant, who has at least 8 hours of contact with the participant each week (e.g., 4 days a week for at least 2 hours per day), and who is available for contact with the study site staff at each visit.

### D. Study Intervention and Concomitant Therapy

### 1. Study Intervention(s) Administered

Seltorexant will be supplied as tablets of 20 mg. Placebo will be supplied as matching tablets. Study drug will be manufactured and provided under the responsibility of the sponsor. Study drug will be provided in blister kits (otherwise described as “container” throughout the document) identified by a study number. Study drug labels will contain information to meet the applicable regulatory requirements.

All participants should take 1 tablet of their assigned study drug once daily at bedtime, with water, with or without a meal, from Day 1 to Day 42.

The tablets must be swallowed whole with water and not chewed, divided, dissolved or crushed. Participants or their study partners are required to record the administration of study drug or any missed doses in participant medication diaries, which will be checked at each scheduled visit. Pill counts of study drug will be performed at in person postbaseline visits during the treatment phase of the study.

If a scheduled (i.e., at bedtime) dose is missed, participants are advised not to take the dose in the morning and not to administer 2 doses at a time the next evening. The dose will be skipped.

### 2. Preparation/Handling/Storage/Accountability

All study drug must be stored at the site at controlled temperatures and conditions as indicated on the product-specific labeling.

### 3. Measures to Minimize Bias: Randomization and Blinding

Central randomization will be implemented in conducting this study for entry to the DB phase. Participants will be randomly assigned based on an algorithm implemented in the IWRS before the study. During the DB phase, participants will be randomly assigned in a 1:1 ratio to receive 1 of 2 treatments: placebo: seltorexant 20 mg.

The randomization will be balanced by using randomly permuted blocks and will be stratified by country (if applicable), NPI-12 sleep domain score (<4, >4), and community dwelling/assisted living. Based on the algorithm, the IWRS will assign a unique treatment code, which will dictate the study drug assignment and matching study drug kit for the participant.

### 4. Study Intervention Compliance

The study drug will be self-administered by the participant at their residence during the DB treatment phase. Dosing and medication diary compliance will be done with oversight and/or assistance by the study partner.

### 5. Continued Access to Study Intervention After the End of the Study

No continued access will be proposed for this study as the long-term safety of seltorexant in this population is unknown.

### 6. Treatment of Overdose

For this study, any dose of seltorexant greater than the number of tablets assigned for each day within a 12-hour time period will be considered an overdose.

In the event of an overdose:

(i) Contact the sponsor's medical monitor or designee immediately.

(ii) Monitor the participant for AEs/SAEs until seltorexant and its M12 metabolite can no longer be detected systemically (at least 3 days).

(iii) Obtain a blood sample for PK analysis within 1 day from the date of the last dose of study drug if requested by the sponsor's medical monitor or designee (determined on a case-by-case basis). The study team will be blinded to the result.

7. Prior and Concomitant Therapy

Modification of a pre-existing medication should not be made for the explicit purpose of entering a participant into the study.

Treatment of stable medical conditions is allowed provided the participants are on stable medication (for at least 30 days prior to screening) and should remain on stable medication, if possible, for the duration of the study. These include but are not limited to:
(i) Stable AChE inhibitors and memantine
(ii) Stable non-sedating antidepressants
(iii) Stable antihypertensives, stable statins, stable anti-thrombotic agents
(iv) Stable treatment for diabetes mellitus for at least 2 months prior to screening
(v) Stable thyroid hormones for at least 3 months prior to screening

Patients who take aducanumab should be on a stable dose/regimen for at least 6 months before screening.

sProhibited Medications

Participants treated with a strong inhibitor of CYP3A4 or CYP2C9 or moderate/strong inducer of CYP3A4 or CYP2C9 or a dual inhibitor/inducer of CYP3A4 and CYP2C9 within 14 days before the first study drug administration on Day 1 or will require treatment during the study must be excluded.

For participants with limited renal (CrCl <60 mL/min) or hepatic disease (AST/ALT >1.5X ULN and bilirubin >1.5X ULN), see table below, moderate CYP3A4 inhibitors or CYP2C9 inhibitors are not allowed within 14 days before the first study drug administration on Day 1 and until the follow-up visit. For participants with limited recent renal or hepatic disease, use the following guidance for study participation and concomitant medication use.

| Renal Function | |
|---|---|
| (CrCl in mL/min) | Impact on Study Participant |
| ≥60 | Eligible for study without restriction |
| 30-59 | Limitation on concurrent |
| | medications |
| <30 | Participant should not be enrolled in the |
| | study (Screen-failure) |

| Hepatic Function | Impact on Study Participant |
|---|---|
| AST/ALT <1.5X ULN and/or | Eligible for study without |
| Bilirubin <1.5X ULN | restriction |
| AST/ALT >1.5X ULN and | Limitation on concurrent drugs, |
| Bilirubin >1.5X ULN | see Table 18 |
| ≥2X ULN) increase of AST or ALT | Participant should not be included |
| at screening (one retest is permitted) | in the study (Screen-failure) |
| Significant liver disease including | |
| cirrhosis, ascites, or active | |
| hepatitis (fatty liver disease and | |
| Gilbert's syndrome will be allowed | |
| as long as the participant does not | |
| meet first criteria) | |

TABLE 18
Examples of Concomitant Drugs to be Avoided (Strong Inhibitors and Moderate/Strong
Inducers of CYP3A4 or CYP2C9 or Dual Inhibitors/Inducers of CYP3A4 and CYP2C9.
In Addition, Moderate CYP3A4 or CYP2C9 Inhibitors for Participants with Creatinine
Clearance <60 mL/min or Clinically Significant Hepatic Disease

| | | | | Dual |
|---|---|---|---|---|
| | | | | Inhibitors or |
| | | | | Inducers of |
| | Inhibitors | Inducers | | CYP3A4 and |
| Enzymes | Strong | Strong | Moderate | CYP2C9 |
| CYP2C9 | None known | None known | rifampin, | fluconazole |
| | | | enzalutamide | rifampin |
| | | | | enzalutamide |
| CYP3A4 | boceprevir | avasimibe | bosentan | |
| | clarithromycin | apalutamide | efavirenz | |
| | indinavir/ritonavir | enzalutamide | etravirine | |
| | itraconazole | mitotane | modafinil | |
| | ketoconazole | carbamazepine | nafcillin | |
| | lopinavir/ritonavir | phenytoin | phenobarbital | |
| | mibefradil | rifampin | primidone. | |
| | nefazodone | St. John's wort | | |
| | nelfinavir | | | |
| | posaconazole | | | |
| | ritonavir | | | |
| | saquinavir/ritonavir | | | |
| | telaprevir/tipranavir/ | | | |
| | ritonavir | | | |
| | telithromycin | | | |
| | voriconazole | | | |
| | idelalisib | | | |
| | cobicistat | | | |
| | danoprevir/ritonavir | | | |
| | elvitegravir/ritonavir | | | |
| | paritaprevir/ritonavir | | | |
| | paritaprevir and | | | |
| | ritonavir and | | | |
| | (ombitasvir and/or | | | |
| | dasabuvir) | | | |
| | troleandomycin | | | |
| | high-dose, double | | | |
| | strength grapefruit juice^{a} | | | |

In addition to the above list, moderate CYP3A4 or CYP2C9 inhibitors for participants with
creatinine clearance <60 mL/min or clinically significant hepatic disease

| | Inhibitors |
|---|---|
| Enzymes | Moderate |
| CYP2C9 | Amiodarone |
| | miconazole |
| | piperine |
| CYP3A4 | Aprepitant |
| | Ciprofloxacin |
| | Conivaptan |
| | Crizotinib |
| | Cyclosporine |
| | Diltiazem |
| | Dronedarone |
| | Erythromycin |
| | Fluvoxamine |
| | Imatinib |
| | Tofisopam |
| | verapamil |

St. John's wort, ephedra, 5-hydroxytryptophan, ashwagandha, Chinese herbal medications known to affect CYP3A4 or CYP2C9, ginkgo, ginseng, or kava from at least 7 days before Day 1 until the duration of the study.

MAOIs should be prohibited by Day-30.

The following medications should be discontinued by Day-7:
Sedating antidepressants: doxepin, trazadone, mirtazapine, agomelatine, and trimipramine, amitriptyline
Mood stabilizers such as Lithium, or anticonvulsants
Benzodiazepines (other than lorazepam as a rescue medication in the protocol defined doses), buspirone, Z-drugs, other hypnotics, sedating antihistamines including over-the-counter hypnotics (e.g., diphenhydramine, doxylamine, and hydroxyzine), and melatonin.
Antipsychotics

When possible, all sleep medication should be stopped within 21 days after signing the ICF (including sedative-hypnotics from the benzodiazepine, non-benzodiazepine and antihistamine classes as well as prazosin, if it is being used for the treatment of sleep problems). Rebound effects of stopping pre-study sleep medication and/or benzodiazepine may be remediated by tapering the medication. An extension of screening by up to 2 weeks may be requested so that the last dose of disallowed medication is at least 7 days prior to baseline/Day 1.

If new medications or changes to medication are required, that are expected to have an impact on study endpoints or safety, these should be initiated following completion of the double-blind treatment phase. If needed to start prior to the end of the DB phase, the initiation of the new medication should be discussed with the Sponsor's medical advisor.

If the participant received an investigational intervention (including investigational vaccines) or used an invasive investigational medical device within 60 days (or 5 half-lives whichever is longest) before the planned first dose of study intervention, or is currently enrolled in an investigational study, they will be excluded from participating in the study.

The following rescue medications may be used:
Lorazepam or equivalent up to 2 mg/day and no more than 3 days out of every 14 days (see Table 17 for benzodiazepine equivalence table)
Rescue medication should not be used the night before or any time prior to assessments on scheduled visit days.

### E. Study Assessments and Procedures

Table 15 summarizes the frequency and timing of efficacy, PK, PD, biomarker, pharmacogenomic, and safety measurements applicable to this study.

### 1. Order of Assessments

Safety assessments, such as vital signs and ECG, are recommended to be performed before blood is drawn and food is provided. On days when fasting laboratory blood samples are taken, it is recommended that efficacy assessments should be administered after food is provided and participants feel comfortable, without help or time pressure, and under quiet conditions.

### 2. Sample Collection and Handling

Refer to Table 15 for the timing and frequency of all sample collections. The total blood volume for the study is approximately 64 mL (30 mL for safety, 4 mL for pharmacokinetics, 6 mL for pharmacogenomics, and 30 mL for biomarkers). For each participant, the maximum amount of blood drawn from each participant in this study will not exceed 120 mL.

### 3. Efficacy Assessments

Assessments should be completed at approximately the same time on each day that testing occurs, and by the same rater(s). The same study partner should complete any caregiver ratings. These procedures should be employed throughout the study to reduce potential variability.

To minimize bias, it is recommended that the raters who conduct the efficacy (NPI-12, NPI-C, SDI, CMAI-C) assessments should not be involved in study drug dosing, physical and neurological evaluation, vital signs, ECG, laboratory and AE evaluation, as well as other safety scale assessments. In this situation, efficacy raters are preferred not to access or review study participant safety records and, therefore, they should not provide clinical care for the study participants.

The following briefly describe the cognitive and functional testing that will be assessed.

### Neuropsychiatric Inventory (NPI-12)

The NPI-12 is a measure of psychobehavioral disturbances, assessing the frequency and severity of disturbances in 12 domains, based on a caregiver interview. For each domain, there is also an assessment of caregiver burden. Frequency for each domain is rated on a 4-point scale (from 1=rarely to 4=very often) and severity on a 3-point scale (from 1=mild to 3=severe), with the score for each domain being the product of the frequency and severity scores, such that each domain is scored from 1 to 12. The NPI-12 total score is the sum of the 12 domain scores, ranging from 0 (best) to 144 (worst). Caregiver distress, based on the response to the question “how emotionally distressing do you find this behavior?”, is rated on a 6-point scale (from 0=not at all to 5=very severely or extremely), with the total score for caregiver distress ranging from 0 (best) to 60 (worst).

Apathy/Indifference, Disinhibition, Irritability/Lability, Aberrant Motor Behavior, Anxiety, Elation/Euphoria, Sleep, and Appetite/Eating Disorders will be assessed using the original NPI-12 domains. These will be based on caregiver interview alone, and the domain's sub-questions will not be asked if the screening question is answered with a “No.”

For Delusions, Hallucinations, Depression/Dysphoria, and Agitation/Aggression domains, regardless of whether the answer to the domain screening question is “Yes” or “No”, the NPI-12 ratings for these domains will be done in their entirety (i.e. —questions asked and rated).

### Neuropsychiatric Inventory Clinician Version (NPI-C)

The NPI-C is an instrument developed on the basis of original NPI that gives a score based on product of frequency and severity ratings of 12 symptom domains that can then be summed to a total score. For NPI-C, the items and domains of the original NPI have been expanded and the rating approach has been modified to include a clinician rating methodology. The rating methodology of the NPI-C incorporates the expert clinician's impressions to the data provided by the patients and caregivers.

The NPI-C can be used to rate the presence of NPS across many domains, as in the NPI-12, as a stand- alone measure for specific NPS domains (e.g., dysphoria, agitation), or a combination of both (presence of NPS across domains plus particular focus on one or more specific domains). Five domains will be measured in the study with NPI-C: Delusions, Hallucinations, Agitation, Aggression, Dysphoria, and will include both caregiver and participant interviews. Regardless of whether the answer to the domain screening question is “Yes” or “No”, the NPI-C ratings for these domains will be done in their entirety (i.e. —questions asked and rated, with a corresponding Clinician Impression of Severity score).

An audio recording will be performed for NPI-C Agitation and Aggression items. The review of the recordings will be conducted by independent centralized raters provided by a study selected vendor for quality purposes.

### Sleep Disorder Inventory (SDI)

The SDI is based on a caregiver interview and an expanded version of one item of the NPI-12. It describes the frequency, severity, and caregiver burden of sleep-disturbed behaviors during a period prior to its administration.

The SDI was created by expanding item 11 of the 12-item NPI. According to the basic structure of the NPI-12, the respondent is asked a ‘screening’ question, a general indication of whether or not symptoms in that particular behavioral area are present. If the screening question is positive, then specific sub-questions are asked. The SDI consists of the seven sub-questions from the NPI-12 sleep disturbance item. Each of the sub-questions was made into a separate question with frequency, severity, and caregiver distress rated by the caregiver with respect to the participant. Thus, in contrast to a single rating for frequency and severity for all sleep disturbance-related behaviors, which would be incorporated into an overall NPI-12 score, the SDI score is derived after the caregiver rates the frequency and severity of each of the seven separate sleep disturbance symptoms. Caregiver distress ratings are not part of the SDI total score, but distress is measured.

Cohen-Mansfield Agitation Inventory—Community version (CMAI-C)

The CMAI-C is a 37-item scale that measures the ability of a drug to reduce overall frequency of agitation symptoms, including aggressive behaviors. Individual items are rated by an expert clinician on a scale of 1 to 7 in which a score of 7 represents the most frequent for each item assessed.

### 4. Safety Assessments

Adverse events will be reported. Any clinically significant abnormalities persisting at the end of the study/early withdrawal will be followed until resolution or until a clinically stable condition is reached. The study will include the following evaluations of safety and tolerability according to the time points provided in Table 15.

### Physical Examination

Height will be measured at screening only. Body weight will be measured for each physical exam according to Table 15.

Body weight should be measured using a calibrated scale at each indicated visit. Participants should be weighed at approximately the same time of day on the same scale, wearing lightweight clothing without shoes; they will be instructed to empty their bladders before being weighed.

### Neurological Examination

The neurological exams will include assessment of sensation, level of alertness, ataxia, tremor, and other routine components of a brief neurological examination.

### Vital Signs

Temperature, pulse/heart rate, and blood pressure will be assessed.

Blood pressure and pulse/heart rate measurements will be assessed in a sitting position with a completely automated device. Manual techniques will be used only if an automated device is not available.

Blood pressure and pulse/heart rate measurements should be preceded by at least 5 minutes of rest in a quiet setting without distractions (e.g., television, cell phones).

### Electrocardiograms

Twelve-lead ECGs, intended for safety monitoring, will be recorded in a supine position so that the different ECG intervals (RR, PR, QRS, QT) can be measured. The ECG will be recorded until 4 regular consecutive complexes are available in good readable quality. If, during screening and/or Day 1, the QTcF is prolonged on the initial ECG at a given time point, the average QTcF of 3 ECGs, recorded 4 minutes apart, must not be ≥450 msec for males and ≥470 msec for females (Exclusion criterion 11).

During the collection of ECGs, participants should be in a quiet setting without distractions (e.g., television, cell phones). Participants should rest in a supine position for at least 5 minutes before ECG collection and should refrain from talking or moving arms or legs. If blood sampling or vital sign measurement is scheduled for the same time point as ECG recording, the procedures are recommended to be performed in the following order: ECG(s), vital signs, blood draw.

### Clinical Safety Laboratory Assessments

Blood samples for serum chemistry and hematology and a random urine sample for urinalysis will be collected. See, Table 19.

TABLE 19

| Laboratory | | | |
|---|---|---|---|
| Assessments | Parameters | | |
| Hematology | Platelet count | RBC Indices: | WBC count with |
| | Red blood cell count | MCV | Differential: |
| | Hemoglobin | MCH | Neutrophils |
| | Hematocrit | % Reticulocytes | Lymphocytes |
| | | | Monocytes |
| | | | Eosinophils |
| | | | Basophils |

| | A WBC evaluation may include any abnormal cells, which will then be | |
|---|---|---|
| | reported by the laboratory. An RBC evaluation may include abnormalities | |
| | in the RBC count, RBC parameters, or RBC morphology, which will then | |
| | be reported by the laboratory. In addition, any other abnormal cells in a | |
| | blood smear will also be reported. | |
| Clinical | Sodium | Total and direct bilirubin |
| Chemistry | Potassium | Alkaline phosphatase |
| | Chloride | CPK |
| | Bicarbonate | LDH |
| | BUN | Uric acid |
| | Creatinine | Calcium |
| | Glucose fasting | Phosphate |
| | Insulin | Albumin |
| | AST/Serum | Total protein |
| | glutamic-oxaloacetic | |
| | ALT/Serum | Cholesterol |
| | glutamic-oxaloacetic | |
| | GGT | Triglycerides |
| | | Magnesium |
| Others | Folate | |
| | B12 | |
| Routine | Dipstick | Sediment (if dipstick |
| Urinalysis | Specific gravity | result is abnormal) |
| | pH | Red blood cells |
| | Glucose | White blood cells |
| | Protein | Epithelial cells |
| | Blood | Crystals |
| | Ketones | Casts |
| | Bilirubin | Bacteria |
| | Urobilinogen | |
| | Nitrite | |
| | Leukocyte esterase | |

| | |
|---|---|
| | If dipstick result is abnormal, flow cytometry will be used to measure |
| | sediment. In case of discordance between the dipstick results and the |
| | flow cytometric results, the sediment will be examined microscopically. |
| | In the microscopic examination, observations other than the presence |
| | of WBC, RBC and casts may also be reported by the laboratory. |
| Other | Urine Drug Screen [opiates (including methadone), cocaine, |
| Screening | amphetamines, methamphetamines, cannabinoids, cannabidiol, |
| Tests | barbiturates, MDMA] |
| | Alcohol breath test |
| | Lipid panel: |
| | Total cholesterol |
| | Triglycerides |
| | Low-density lipoprotein cholesterol |
| | High-density lipoprotein cholesterol |
| | HbA1c |
| | TSH. For participants with a history of thyroid disease and for |
| | participants who, regardless of thyroid history have TSH value out |
| | of range, a FT₄ test will be conducted |
| | Plasma concentrations of seltorexant, its M12 metabolite, and AGP. |
| | Additional drug and alcohol tests may be conducted as needed per the |
| | investigator's judgement |

### Suicidal Ideation and Behavior Risk Monitoring

Seltorexant is considered to be a CNS-active medication being developed for MDD with insomnia symptoms and BPSD in Patients with probable Alzheimer's Disease. There has been some concern that CNS-active medications may be associated with an increased risk of suicidal ideation or behavior. Although this study intervention or other similar treatments in this class have not been shown to be associated with an increased risk of suicidal thinking or behavior when given to healthy volunteers or patients, the sponsor considers it important to monitor for such events before or during this clinical study.

Emergence of potential suicidal ideation will be assessed using the C-SSRS clinician version at screening, and at all subsequent study visits. The C-SSRS is a low-burden measure of the spectrum of suicidal ideation and behavior that was developed to assess severity and track suicidal events through any treatment. It is a clinical interview providing a summary of both suicidal ideation and behavior that can be administered during any evaluation or risk assessment to identify the level and type of suicidality present. The C-SSRS has been used frequently in clinical studies, and is a validated, standard measure for suicidal ideation assessment.

Two versions of the C-SSRS will be used in this study, the Baseline/Screening version, and the Since Last Visit version. The Baseline/Screening version of the C-SSRS will be used at the screening visit. In this version, suicidal ideation will be assessed at 2 time points (“lifetime” and “in the past 6 months”) and suicidal behavior will be assessed at 2 time points (“lifetime” and “in the past year”).

Participants are excluded if they have serious suicidal ideation (corresponding to a positive response to C-SSRS item 4 or 5) within 3 months or suicidal behavior within 6 months of study entry.

All subsequent C-SSRS assessments in this study will use the Since Last Visit version, which will assess suicidal ideation and behavior since the participant's last visit.

### Alzheimer's Disease Assessment Scale Cognitive Subscale 14-Item Version (ADAS-Cog-14)

The ADAS-Cog is a rater administered instrument that was designed to assess the severity of dysfunction in cognition characteristic of persons with AD. The ADAS-Cog11 consists of 11 tasks measuring the disturbances of memory, language, praxis, attention, and other cognitive abilities, which are often referred to as the core symptoms of AD.

The modified ADAS-Cog 14-item scale includes all original ADAS-Cog items with the addition of a number cancellation task, Maze task and a delayed free recall task, for a total of 90 points, with higher scores indicative of worse cognitive performance.

### MMSE

The MMSE test is a 30-point questionnaire that is used extensively in clinical and research settings to measure cognitive impairment. It is commonly used in medicine and allied health to screen for dementia. The test is divided into two sections: the first section requires vocal responses and covers orientation, memory, and attention. The second part tests ability to name, follow verbal and written commands, write a sentence spontaneously, and copy a complex polygon similar to a Bender-Gestalt Figure. The score ranges from 0 (minimum score) to 30 (maximum score) and it is calculated by the sum of the sub-items scored 0 (incorrect answer) or 1 (correct answer).

### Physician Withdrawal Checklist (PWC)

Potential withdrawal effects will be assessed by the PWC-20 according to Table 15. The PWC-20 is a simple and accurate method used to assess potential withdrawal symptoms following cessation of treatment. The PWC-20 is a reliable and sensitive instrument for the assessment of discontinuation symptoms.

### 5. Other Assessments and Procedures

### Clinical Dementia Rating (CDR)

The CDR is a structured clinician-rated interview that serves as a global clinical staging instrument that includes 3 cognitive and 3 functional ratings, including: 1) memory, 2) orientation, 3) judgment and problem solving, 4) involvement in community affairs, 5) home and hobbies, and 6) personal care based on the CDR interview. The CDR has been used as a global clinical staging measure in a number of AD trials. The CDR is scored 2 ways yielding a global CDR score (CDR-GS, derived from an algorithm developed by the Knight Alzheimer Disease Research Center at Washington University School of Medicine in St. Louis, Missouri, US, and including categorical scoring of 0, 0.5, 1, 2, and 3), as well as CDR-SB (the continuous sum of 6 domains, up to a total score of 18, with higher scores representing worse disease state).

### Rosen Modified Hachinski Ischemic Scale (HIS)

The HIS is a clinical questionnaire collecting information relevant for the differentiation between the most common dementia types, Dementia of Alzheimer's Type (DAT) and vascular dementia. The Rosen Modified HIS calculates the likelihood dementia is due to vascular causes. Because it systematically identifies risk factors for vascular cognitive impairment, it is a relevant screening instrument for earlier stages of AD and will be performed at screening. A cutoff score ≤4 for DAT and ≥7 for vascular dementia has a sensitivity of 89% and a specificity of 89%.

### Brief Psychosocial Therapy

Brief psychosocial therapy offered in the study was specifically developed for use in clinical trials of Alzheimer's patients with behavioral disorders. The therapy is a non-pharmacological intervention that will be used during the screening period. It is simple to use and involves regular interactions between a caregiver (study partner) and the participant based on a plan designed specifically for them. The important aspect of the brief psychosocial therapy is to ensure the participants receive quality social interaction that they find interesting and engaging.

The brief psychosocial therapy entails daily 10-15 minute semi-structured interactions between the participant and a designated caregiver (study partner). The study partner will require training and assistance from site staff in developing and implementing an individually-designed interaction strategy for the participant involved. All site staff therapists in the study will complete therapist qualification training prior to commencing work on the study.

There will be approximately four visits between the site staff (therapist) and the study partner during the screening period (phone and site visits) leading up to baseline and randomization. The purpose of each visit is summarized below:

Training Visit: In-person brief psychosocial therapy training for the study partner occurs in the clinic at this first study visit (Screening Visit 1). This session is specifically designed to help the therapist and study partner understand each participant and design an individualized plan of interaction documented in a BPST-AD Treatment Plan. This session will last between 45 and 60 minutes.

Follow-up Visits: Follow-up telephone contacts may occur approximately every 4 to 5 days after the Training Visit at Screening. The study partner is contacted by the site staff therapist to assess how the brief psychosocial therapy is progressing, to review the caregiver worksheet completed by the study partner and to provide the study partner with supportive suggestions for any concerns. At least one phone follow-up visit should be conducted during the screening period for all participants.

Baseline Visit: This in-person visit occurs at the clinic at the conclusion of the screening period (Baseline Visit 2), at least two weeks after the Training Visit conducted at Screening. Then the brief psychosocial therapy intervention is formally closed.

### Objective Sleep and Physical Activity Parameters (Actigraphy)

Actigraphy is the process of measuring physical movement of an individual over time to assess the degree of motor activities and will provide an objective measure of sleep prior to treatment. The actigraphy device is a small, motion biosensor that is worn on the non-dominant wrist to monitor daily activity and sleep patterns. Participants are asked to wear the device continuously throughout the screening and DB phases of the study. Actigraphy recorded movements can be used to estimate sleep parameters with specialized algorithms. Actigraphy has been validated for the estimation of nighttime sleep parameters across age groups and psychiatric conditions. Subjective and objective sleep assessment may assess different constructs of sleep and may not have a high correlation.

Participants will be instructed to wear an actigraphy device on their non-dominant wrist from screening to end of the DB phase or early withdrawal. Participants should wear it during the day and night except when bathing. If a participant is not able to tolerate wearing the actigraphy device (either at night or during the day), the participant may continue in the study and the use of the actigraphy device may be discontinued. Movements during the day and night will be captured. Daytime physical activity may be analyzed. The primary purpose of actigraphy in this study is to have an objective measure of sleep and physical activity parameters to complement the SDI, CMAI-C, NPI-12 and NPI-C.

The parameters collected will include but are not limited to the following:
SOL
TST
WASO
sleep efficiency
physical activity.

### Off-Body Sensors

This procedure will be implemented in the study as an optional assessment and only if feasible. The off-body sensors will be used in a subset of participants. Participants or their study partner may opt-out of using the off-body sensors. The sensor(s) will be placed in the participants home and used from screening to end of treatment.

### 6. Adverse Events, Serious Adverse Events, and Other Safety Reporting

An AE is any untoward medical occurrence in a clinical study participant administered a pharmaceutical (investigational or non-investigational) product. An AE does not necessarily have a causal relationship with the intervention. An AE can therefore be any unfavorable and unintended sign (including an abnormal finding), symptom, or disease temporally associated with the use of a medicinal (investigational or non-investigational) product, whether or not related to that medicinal (investigational or non-investigational) product. (Definition per ICH)

This includes any occurrence that is new in onset or aggravated in severity or frequency from the baseline condition, or abnormal results of diagnostic procedures, including laboratory test abnormalities.

A SAE based on ICH and EU Guidelines on Pharmacovigilance for Medicinal Products for Human Use is any untoward medical occurrence that at any dose:
Results in death
Is life-threatening; The participant was at risk of death at the time of the event. It does not refer to an event that hypothetically might have caused death if it were more severe.
Requires inpatient hospitalization or prolongation of existing hospitalization
Results in persistent or significant disability/incapacity
Is a congenital anomaly/birth defect
Is a suspected transmission of any infectious agent via a medicinal product
Is Medically Important

If a serious and unexpected AE occurs for which there is evidence suggesting a causal relationship between the study intervention and the event (e.g., death from anaphylaxis), the event must be reported as a serious and unexpected suspected adverse reaction even if it is a component of the study endpoint (e.g., all-cause mortality).

### Severity Criteria

An assessment of severity grade will be made using the following general categorical descriptors:

Mild: Awareness of symptoms that are easily tolerated, causing minimal discomfort and not interfering with everyday activities.

Moderate: Sufficient discomfort is present to cause interference with normal activity.

Severe: Extreme distress, causing significant impairment of functioning or incapacitation. Prevents normal everyday activities.

The investigator should use clinical judgment in assessing the severity of events not directly experienced by the participant (e.g., laboratory abnormalities).

### All Adverse Events

All AEs and special reporting situations, whether serious or non-serious, will be reported from the time a signed and dated ICF is obtained until completion of the participant's last study-related procedure, which may include contact for follow-up of safety.

### Serious Adverse Events

All SAEs occurring during the study must be reported.

Serious adverse events, including those spontaneously reported within 30 days after the last dose of study intervention, must be reported. The sponsor will evaluate any safety information that is spontaneously reported beyond the time frame specified in the protocol.

### Pregnancy

All initial reports of pregnancy in partners of male participants must be reported to the sponsor by the study-site personnel within 24 hours of their knowledge of the event using the appropriate pregnancy notification form. Abnormal pregnancy outcomes (e.g., spontaneous abortion, fetal death, stillbirth, congenital anomalies, ectopic pregnancy) are considered SAEs.

Because the effect of the study drug on sperm is unknown, pregnancies in partners of male participants included in the study will be reported as noted above.

Follow-up information regarding the outcome of the pregnancy and any postnatal sequelae in the infant will be required.

### Adverse Events of Special Interest

The following AEs are considered to be of special interest in this study:
Cataplexy (sudden, transient episode of muscle weakness accompanied by conscious awareness)
Sleep paralysis (the experience of not being able to move, react, or speak when falling asleep/awakening)
Complex, sleep-related behaviors/parasomnias such as confusional arousals, somnambulism (sleep walking), sleep terrors, bruxism (teeth grinding), sleep sex, sleep related eating disorder, and catathrenia (REM-associated end-inspiratory apnea/breath holding)
Fall (defined as an event which results in a person coming to rest inadvertently on the ground or floor or other lower level; falling, loss of posture, falling down).

### 7. Pharmacokinetics

Plasma samples will be used to evaluate the PK of seltorexant. Plasma collected for PK may additionally be used to evaluate safety or efficacy aspects that address concerns arising during or after the study period. Genetic analyses will not be performed on these plasma samples.

### Evaluations

Blood samples for the determination of plasma concentrations of seltorexant its M12 metabolite, and alpha-1-acid glycoprotein, will be collected from participants per Table 15.

In addition, blood samples will be collected for determination of plasma concentrations of seltorexant, its M12 metabolite, and alpha-1-acid glycoprotein in participants who discontinue study drug for an AE, have an AESI, or have an SAE if the sample can be obtained within 15 hours of the last study drug administration.

Blood samples will be collected to determine alpha-1-acid glycoprotein levels at each PK collection day (Table 15) to calculate the unbound concentrations.

During the DB phase, blood samples for PK will be collected from all participants, including placebo-treated participants, but samples from placebo-treated participants will not be analyzed for PK. These samples will be stored and may be analyzed if needed (e.g., suspicion of an incorrect treatment assignment).

### Analytical Procedures

Plasma samples will be analyzed to measure concentrations of seltorexant and its M12 metabolite using a validated, specific, and sensitive LC-MS/MS method.

AGP levels will be determined.

### Pharmacokinetic Parameters and Evaluations

Plasma concentration-time data will be displayed by visit date and time for seltorexant and its M12 metabolite. The alpha-1-acid glycoprotein levels will be tabulated for each participant.

### 7. Genetic and Pharmacogenomic Evaluations

Blood samples will be collected from participants who consent separately to this component of the study (where local regulations permit) to allow for genotyping of the CYP2C9 and CYP3A4 genes, which may influence metabolism of seltorexant. Samples will also be assessed for APOE genotype.

### 8. Biomarkers

Biomarker blood samples will be collected to explore biomarkers related to AD, including but not limited to: p217+tau, A042/40 levels and ratio, and NfL.

Biomarker assays may be added or deleted based on scientific information or technical innovations under the condition that the total volume of blood collected will not be increased.

Biomarker analyses are dependent upon the availability of appropriate biomarker assays and clinical response rates. Biomarker analysis may be deferred or not performed, if during or at the end of the study, it becomes clear that the analysis will not have sufficient scientific value for biomarker evaluation, or if there are not enough samples or responders to allow for adequate biomarker evaluation. In the event the study is terminated early or shows poor clinical efficacy, completion of biomarker assessments is based on justification and intended utility of the data.

### F. Statistical Considerations

Statistical analysis will be done by the sponsor or under the authority of the sponsor. A general description of the statistical methods to be used to analyze the efficacy and safety data is outlined below.

### 1. Statistical Hypotheses

This study is designed to show that the treatment effect in improving agitation and aggression (as measured by change from baseline on Day 43 in NIPC A+A score) of seltorexant 20 mg is superior to placebo in participants with probable AD with clinically significant agitation/aggression.

If μ_{T} is the mean change in NPI-C A+A for seltorexant 20 mg group and pp is the mean change in NPC A+A for the placebo group, then the hypothesis can be written as follows:

H₀: μ_{T}−μ_{P}≥0 vs.

H₁: μ_{T}−μ_{P}<0

Superiority in seltorexant 20 mg can be concluded if the one-sided p-value for the testing of the hypothesis above is less than 0.1 (equivalently, the two-sided p-value for the testing of the hypothesis above is less than 0.2, with greater improvement in the 20 mg group).

### 2. Sample Size Determination

Assuming a treatment difference of 5 points in change from baseline in NPI-C A+A score between seltorexant 20 mg and placebo, a standard deviation of 10 (effect size of 0.5), 15% drop-out rate during the 6-week double-blind phase, 86 participants (randomized in 1:1 ratio to placebo and seltorexant 20 mg) will need to be enrolled in the double-blind phase, in order to achieve 80% power to detect treatment difference, at one-sided 0.1 significance level.

### 3. Populations for Analysis Sets

For purposes of analysis, the following analysis sets are defined:

| Analysis Set | Description |
|---|---|
| Screened | All participants who sign the ICF |
| Randomized | All participants who were randomized in the study |
| Full | All randomized participants who take at least 1 |
| | dose of study intervention |
| Safety | All randomized participants who take at least 1 |
| | dose of study intervention. |

### 4. Statistical Analyses

For all efficacy endpoints, descriptive statistics of the actual values and the change from baseline to each postbaseline time point in the double-blind phase will be presented by intervention group.

### Primary Endpoint(s)

The analyses of efficacy endpoints will be based on the full analysis set.

The primary efficacy endpoint is the change in NPI-C A+A score from baseline to Day 43.

There are two estimands defined for the primary efficacy endpoint. The primary estimand is Estimand 1, and the supplementary estimand is Estimand 2.

### Estimand 1:

Study Intervention:
Experimental: seltorexant
Control: placebo

Population: participants with probable AD with clinically significant agitation/aggression, as reflected by the inclusion/exclusion criteria.

Variable: change in NPI-C A+A from baseline to Day 43.

Summary measure: difference in treatment means between placebo and 20 mg.

Intercurrent events and corresponding strategies:
Treatment discontinuation of study drug (Hypothetical strategy: as if the intercurrent event had not occurred)
Switch of treatment (Hypothetical strategy: see above)
Allowed rescue medication (Treatment policy strategy: all observed values of the endpoint are used regardless of whether or not the participant had experienced this intercurrent event)

### Estimand 2:

All components are the same as Estimand 1, except for intercurrent events and corresponding strategies.

Intercurrent events and corresponding strategies:
Treatment discontinuation of study drug (Treatment policy strategy: all observed values of the endpoint are used regardless of whether or not the participant had experienced this intercurrent event)
Switch of treatment (Hypothetical strategy: as if the intercurrent event had not occurred)
Allowed rescue medication (Treatment policy strategy: all observed values of the endpoint are used regardless of whether or not the participant had experienced this intercurrent event)

### Main Analysis Under Estimand 1

The comparison between seltorexant 20 mg and placebo will be performed using the appropriate contrasts in a MMRM with main comparison at Day 43. The MMRM will include stratification factors, time, intervention group (placebo and seltorexant 20 mg) and time-by-intervention interaction as factors, and baseline NPI-C A+A as a covariate. Difference in least square means and 2-sided 80% CI will be presented.

### Main Analysis Under Estimand 2

The copy reference (CR) multiple imputation (MI) method will be performed. A mixed model (which will include the same factors and covariates as in the main analysis for Estimand 1) will be applied to each imputed dataset (with the CR MI method), and the Rubin's rule will be used to combine results from each imputed dataset.

### Safety Analyses

All safety analyses will be made on the Safety Analysis Set.

### Adverse Events

Treatment-emergent adverse events are AEs with onset during the DB treatment phase or that are a consequence of a pre-existing condition that has worsened since baseline. All reported treatment-emergent AEs will be included in the analysis. For each AE, the percentage of participants who experience at least 1 occurrence of the given event will be summarized by intervention group.

Summaries, listings, datasets, or participant narratives may be provided, as appropriate, for those participants who die, who discontinue intervention due to an AE, or who experience a severe or an SAE or an AESI.

### Clinical Laboratory Tests

Laboratory data will be summarized by type of laboratory test. Reference ranges and markedly abnormal results will be used in the summary of laboratory data. Descriptive statistics will be calculated for each laboratory analyte at baseline and for observed values and changes from baseline at each scheduled time point. Frequency tabulations of the laboratory abnormalities will be made. A listing of participants with any markedly abnormal laboratory results will be provided.

### Electrocardiogram

The effects on ECG measurements (heart rate, PR interval, QT interval, and QTc interval) will be evaluated using descriptive statistics and frequency tabulations. QTc intervals will be calculated using the Bazett and Fridericia correction methods and summarized accordingly.

Descriptive statistics of QTc intervals and changes from baseline will be summarized at each scheduled time point. The percentage of participants with QTc interval higher than pre-specified levels will be summarized, as will the percentage of participants with QTc interval increases from baseline >30 milliseconds or >60 milliseconds.

### Vital Signs

Descriptive statistics of pulse, sitting blood pressure (systolic and diastolic), and temperature for observed values will be provided and changes from baseline will be summarized at each scheduled time point by intervention group. The percentage of participants with values beyond clinically important limits will be summarized. Changes in body weight, waist circumference, and BMI will be summarized descriptively.

### Physical and Neurological Examinations

Participants with abnormal findings in physical or neurological examination will be presented in a data listing.

### C-SSRS

Suicide-related thoughts and behaviors based on the C-SSRS will be summarized by intervention group.

### Cognitive Symptoms

Descriptive statistics of observed values and change from baseline over time will be provided for ADAS-Cog and MMSE by intervention group.

### Withdrawal Effects (PWC-20)

For PWC-20, frequency for individual item score and mean summaries for a total score (based on 8 items) will be provided by intervention group.

### Pharmacokinetic Analyses

Concentration-time data will be displayed by visit date, and time for seltorexant and metabolite, M12. The levels of al-acid glycoprotein will also be tabulated. In addition, plasma concentrations of seltorexant, its M12 metabolite, and al-acid glycoprotein in participants who discontinue study intervention for an AE, have an AESI, or have an SAE if the sample can be obtained within 15 hours of the last dose will be tabulated.

### Population PK Modelling

If feasible, a population PK analysis using a developed population PK model based on a selection of clinical studies will be performed at the completion of the study. Using actual sampling and dosing times, concentration time data will be analyzed using population PK modeling. Empirical Bayes estimates (i.e., individual PK parameters estimates) will be used to derive individual estimates of the total and unbound exposure parameters (e.g., AUC) for seltorexant, and metabolite M12. As part of the population PK modeling, the effect of intrinsic (e.g., age, gender, body weight) and extrinsic factors (e.g., concomitant medications) affecting the PK of seltorexant may be evaluated if needed. The results of the population PK analysis will be reported separately. The al-acid glycoprotein levels will be tabulated for each participant by visit date and time and will be used to estimate the Fu and predict the unbound concentrations and estimate the individual unbound exposure parameters for seltorexant and major metabolite M12.

A snapshot date for PK samples to be analyzed will be defined, if required. Samples collected before this date will be analyzed for seltorexant and included in the population PK analysis. Samples collected after the snapshot date will be analyzed at a later date, and may be included in a population PK re-analysis when they become available after database lock.

Data will be listed for all participants with available plasma concentrations per intervention group. Participants will be excluded from the PK analysis if their data do not allow for accurate assessment of the PK (e.g., incomplete administration of the study intervention; missing information of dosing and sampling times; concentration data not sufficient for PK parameter calculation).

All concentrations below the lowest quantifiable concentration or missing data will be labeled as such in the concentration database. All participants and samples excluded from the analysis will be clearly documented in the study report.

### Biomarker, Genetic and Pharmacogenomic Analyses

Biomarker endpoints will be summarized, including change from baseline plasma concentrations of p217+tau, Aβ42/40 levels and ratio, NfL, and other exploratory markers.

DNA samples will be analyzed for APOE genotype and to allow for the possible evaluation of genetic factors that may influence the PK, PD, safety, or tolerability of seltorexant and pathways related to AD.

All biomarker and genetic data obtained during this study may be included in ongoing cross-study analyses to investigate the relationship between phenotypes and biomarkers, or to help explain inter-individual variability in clinical outcomes or safety.

Genetic research on enrolled participants may also consist of the analysis of CYP2C9 and CYP3A4/5 in relation to seltorexant.

Biomarker and genetic analyses are dependent upon the availability of appropriate assays and clinical response rates. Biomarker and genetic analysis may be deferred or not performed, if during or at the end of the study, it becomes clear that the analysis will not have sufficient scientific value for biomarker or genetic evaluation, or if there are not enough samples or responders to allow for adequate biomarker or genetic evaluation. In the event the study is terminated early or shows poor clinical efficacy, completion of biomarker or genetic assessments is based on justification and intended utility of the data. Results may be presented in a separate report.

### Pharmacokinetic/Pharmacodynamic Analyses

PK and efficacy assessments relationship may be explored, if feasible.

The disclosures of each patent, patent application, and publication cited or described in this document are hereby incorporated herein by reference, in its entirety.

Those skilled in the art will appreciate that numerous changes and modifications can be made to the preferred embodiments of the disclosure and that such changes and modifications can be made without departing from the spirit of the disclosure. It is, therefore, intended that the appended claims cover all such equivalent variations as fall within the true spirit and scope of the disclosure.

### ASPECTS OF THE INVENTION

1. A method of treating agitation in a human patient with dementia, comprising administering to the human patient with dementia in need thereof a pharmaceutically effective amount of a compound of formula (I):

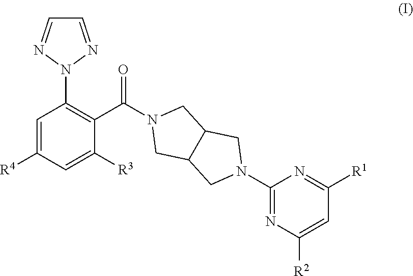

wherein:
   R¹ is C₁₋₄ alkyl;
   R² is C₁₋₄ alkyl;
   R³ is H or halogen; and
   R⁴ is H or C₁₋₄ alkoxy:
or a pharmaceutically acceptable salt or hydrate thereof to treat agitation wherein there is at least a 20% reduction in patient agitation relative to the patient before administration of the compound of formula (I).
2. The method of aspect 1 wherein there is at least a 30% reduction in patient agitation relative to the patient before administration of the compound of formula (I).
3. The method of aspect 1 wherein the dementia is selected from the group consisting of Alzheimer's dementia, frontotemporal dementia, dementia with Lewy Bodies, poststroke dementia, and mixed Alzheimer's disease/vascular dementia.
4. The method of aspect 3 wherein the Alzheimer's dementia is mild or moderate dementia.
5. The method of any one of the preceding aspects, wherein the compound is [5-(4,6-dimethyl-pyrimidin-2-yl)-hexahydro-pyrrolo[3,4-c]pyrrol-2-yl]-(2-fluoro-6-[1,2,3]triazol-2-yl-phenyl)-methanone or a pharmaceutically acceptable salt thereof
6. The method of any one of aspects 1-4, wherein the compound is (5-(4,6-dimethylpyrimidin-2-yl)hexahydropyrrolo[3,4-c]pyrrol-2(1H)-yl)(4-methoxy-2-(2H-1,2,3-triazol-2-yl)phenyl)methanone or a pharmaceutically acceptable salt thereof.

What is claimed is:

1. A method of treating agitation in a human patient with dementia, comprising administering to the human patient with dementia in need thereof a pharmaceutically effective amount of a compound that is:
[5-(4,6-dimethyl-pyrimidin-2-yl)-hexahydro-pyrrolo[3,4-c]pyrrol-2-yl]-(2-fluoro-6-[1,2,3]triazol-2-yl-phenyl)-methanone: 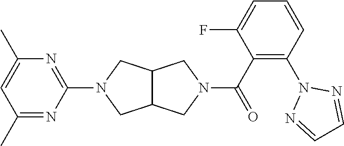 [SMILES: Cc1cc(C)nc(N2CC3CN(C(=O)c4c(F)cccc4-n4nccn4)CC3C2)n1]
or a pharmaceutically acceptable salt or hydrate thereof,
or
(5-(4,6-dimethylpyrimidin-2-yl)hexahydropyrrolo[3,4-c]pyrrol-2(1H)-yl)(4-methoxy-2-(2H-1,2,3-triazol-2-yl)phenyl)methanone: 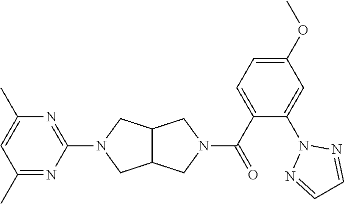 [SMILES: COc1ccc(C(=O)N2CC3CN(c4nc(C)cc(C)n4)CC3C2)c(-n2nccn2)c1]
or a pharmaceutically acceptable salt or hydrate thereof.

2. The method according to claim 1, wherein there is at least a 20% reduction in patient agitation relative to the patient before administration of the compound of formula (I).

3. The method according to claim 2, wherein there is at least a 30% reduction in patient agitation relative to the patient before administration of the compound of formula (I).

4. The method according to claim 1, wherein the dementia is selected from the group consisting of Alzheimer's dementia, frontotemporal dementia, dementia with Lewy Bodies, poststroke dementia, and mixed Alzheimer's disease/vascular dementia.

5. The method according to claim 4, wherein the dementia is Alzheimer's dementia.

6. The method according to claim 5, wherein the Alzheimer's dementia is mild or moderate dementia.

7. The method according to claim 1, wherein the treatment for agitation prevents the escalation to aggression.

8. The method according to claim 1, wherein the treatment improves agitation and aggression.

9. The method according to claim 8, wherein the improvement in agitation and aggression is measured by the NPI-C A+A score.

10. The method according to claim 9, wherein the improvement in agitation and aggression is measured by a change from baseline to a second time point in the NPI-C A+A score.

11. The method according to claim 1, wherein the compound is [5-(4,6-dimethyl-pyrimidin-2-yl)-hexahydro-pyrrolo[3,4-c]pyrrol-2-yl]-(2-fluoro-6-[1,2,3]triazol-2-yl-phenyl)-methanone or a pharmaceutically acceptable salt or hydrate thereof

12. The method according to claim 11, wherein the compound is [5-(4,6-dimethyl-pyrimidin-2-yl)-hexahydro-pyrrolo[3,4-c]pyrrol-2-yl]-(2-fluoro-6-[1,2,3]triazol-2-yl-phenyl)-methanone or a pharmaceutically acceptable salt thereof

13. The method according to claim 1, wherein the compound is (5-(4,6-dimethylpyrimidin-2-yl)hexahy dropyrrolo[3,4-c]pyrrol -2(1H)-yl)(4-methoxy -2-(2H-1,2,3-triazol-2-yl)phenyl)methanone or a pharmaceutically acceptable salt or hydrate thereof.

14. The method according to claim 13, wherein the compound is (5-(4,6-dimethylpyrimidin-2-yl)hexahydropyrrolo[3,4-c]pyrrol-2(1H)-yl)(4-methoxy-2-(2H-1,2,3-triazol-2-yl)phenyl)methanone or a pharmaceutically acceptable salt thereof

15. The method according to claim 1, wherein the effective amount of the compound is about 10 to about 60 mg.

16. The method according to claim 15, wherein the effective amount of the compound is about 20 mg.
